(19) United States
(12) Patent Application Publication
(10) Pub. No.: US 2025/0195699 A1
(43) Pub. Date: Jun. 19, 2025

(54) Radiolabeled Mu Opioid Antagonist and Methods of Making and Using the Same

(71) Applicant: REGENTS OF THE UNIVERSItY OF MICHIGAN, Ann Arbor, MI (US)

(72) Inventors: Jason Witek, Ann Arbor, MI (US); John Traynor, Ann Arbor, MI (US); Peter J. H. Scott, Ypsilanti, MI (US)

(21) Appl. No.: 18/981,182

(22) Filed: Dec. 13, 2024

Related U.S. Application Data
(60) Provisional application No. 63/609,827, filed on Dec. 13, 2023.
(60) Provisional application No. 63/656,414, filed on Jun. 5, 2024.

(51) Int. Cl. A61K 51/04; C07D 405/12; C07D 405/14

(52) U.S. Cl. CPC A61K 51/0455; C07D 405/12; C07D 405/14

(57) ABSTRACT

Disclosed herein are radiolabeled compounds that can act as antagonists of the μ-opioid receptor, including methods of making and using the same. The selective mu opioid receptor antagonists disclosed herein can improve safety for subjects as well as enable studies of the μ-opioid receptor using agonist/antagonist radioligand pairs.

### CROSS-REFERENCE TO RELATED APPLICATIONS

The benefit of priority to U.S. Provisional Patent Application Nos. 63/657,414 filed Jun. 7, 2024, and 63/609,827 filed Dec. 13, 2023, is hereby claimed, and the respective disclosures are incorporated herein by reference in their entireties.

### FIELD

The present disclosure relates generally to compounds that act as antagonists of the μ-opioid receptor and their use in imaging modalities.

### BACKGROUND

Opioid receptors (mu, delta, kappa and nociceptin/orphanin FQ peptide) play a key role in the mechanism of action of both synthetic and natural analgesics. Since the 1970s, PET imaging of opioid receptors has provided a useful tool to understand the role of opioid receptors in pain research as well as in psychiatric and neurological disorders. It is known that opioid receptors exist in a plethora of conformational states and that agonists and antagonists bind differently to them. More specifically, agonists have a high affinity for active receptors coupled to effector G-proteins, but low affinity for uncoupled, inactive receptors while antagonists bind with the same affinity regardless of the conformational state.

Further understanding of the opioid system could be accomplished through the use of agonist/antagonist pairs of opioid PET tracers and studies related to differences in how they bind. However, to date there is still a need for the design and development of new opioid PET tracers that are agonist/antagonist pairs, especially ones that bind the less explored kappa and nociceptin/orphanin FQ opioid receptors.

### SUMMARY

Currently, the radioligand of choice for studies related to the μ-opioid receptor is an agonist and presents safety issues because of its extremely high potency. Selective mu opioid receptor antagonists, such as those of the present disclosure, can improve safety for subjects as well as enable studies of the μ-opioid receptor using agonist/antagonist radioligand pairs.

Provided herein are compounds, or pharmaceutically acceptable salts thereof, having a structure of Formula (I)

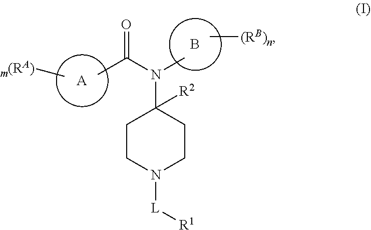

wherein: each m and n are independently 0, 1, 2, or 3; ring A is a 4-8 membered heterocycle, or 5-10 membered heteroaryl, wherein the heterocycle and heteroaryl each comprises 1, 2, or 3 ring heteroatoms selected from N, O, and S; each R^{A}, when present, is C₁₋₃alkyl or C₁haloalkyl; ring B is a C₆₋₁₀aryl, or 5-10 membered heteroaryl, wherein the heteroaryl comprises 1, 2, or 3 ring heteroatoms selected from N, O, and S; each R^{B}, when present, is C₁₋₃alkyl or C₁haloalkyl; L is C₂₋₆alkylene; R¹ is halo, C₁₋₆alkoxyl, C₁₋₆haloalkyl, C₁₋₆haloalkoxyl, C₆₋₁₀aryl, 4-8 membered heterocycle, or 5-10 membered heteroaryl, wherein the heterocycle and heteroaryl each comprises 1, 2, or 3 ring heteroatoms selected from N, O, and S, and when R¹ is C₆₋₁₀aryl, 4-8 membered heterocycle or 5-10 membered heteroaryl, R¹ is substituted with 1 to 3 R^{1A} groups; each R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X; R² is H, —C(O)O—C₁₋₃alkyl, —C(O)O—C₁₋₃alkylene—X, or —C(O)O—C₁₋₃alkylene—LG; X is a radioisotope; and LG is a leaving group; with the proviso that only one X is present or X is absent.

Also provided herein are compounds, or pharmaceutically acceptable salt thereof, having a structure of Formula (II)

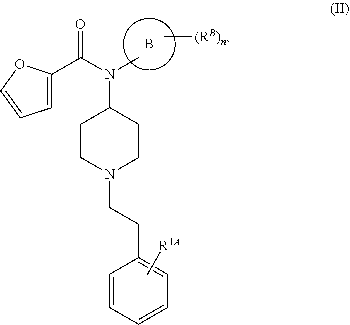

wherein: n is 0 or 1; ring B is phenyl or pyridyl; each R^{B}, when present, is C₁alkyl or C₁haloalkyl; R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X, X is a radioisotope; and LG is a leaving group with the proviso that exclusively one X is present.

Further provided herein are methods comprising administering to a subject the compound of Formula (I) or Formula (II); and subjecting the subject to an imaging modality.

Further aspects and advantages will be apparent to those of ordinary skill in the art from a review of the following detailed description. The description hereafter includes specific embodiments with the understanding that the disclosure is illustrative, and is not intended to limit the invention to the specific embodiments described herein.

### BRIEF DESCRIPTION OF THE DRAWINGS

FIG. 1 shows selected known opioid receptor agonists and antagonists of the μ-, δ-, and κ-opioid receptors.

FIG. 2 shows examples of known fentanyl core-containing antagonists.

Scheme 1 shows the general synthetic route for compounds of the disclosure.

Scheme 2 shows the synthetic route for Boc amine compound 1.

Scheme 3 shows the synthetic route for Boc amide compound 2.

Scheme 4 shows the synthetic route for secondary amine 4.

Scheme 5 shows the synthesis of a radiochemical precursor, Fentanyl Analog Hydroxy Precursor 7.

Scheme 6 shows the synthesis of a radiochemical precursor, 3-Me3SnFN (2).

Scheme 7 shows the automated radiosynthesis of [18F]3FN

FIG. 3A shows a chromatograph of [18F]3FN with impurities.

FIG. 3B shows a chromatograph of [18F]3FN after purification.

FIG. 4A is an image of a pituitary gland in a monkey brain as control.

FIG. 4B is an image of a pituitary gland in a monkey brain showing uptake of 18F]3FN after 15 minutes.

FIG. 5A is an image of a monkey brain, 15 minutes after administration of [18F]3FN.

FIG. 5B is an image of a monkey brain after 15 minutes, as control.

FIG. 5C is an image of a monkey brain, after administration of [18F]3FN.

FIG. 5D is an image of a monkey brain for control.

### DETAILED DESCRIPTION

Provided herein are radiolabeled opioid analogues, as well as methods of making and using the same. The radiolabeled opioid analogues in accordance with the disclosure can be fentanyl analogues or carfentanil analogues. For example, the radiolabeled opioid analogues can be labeled with ¹⁸F or ¹¹C.

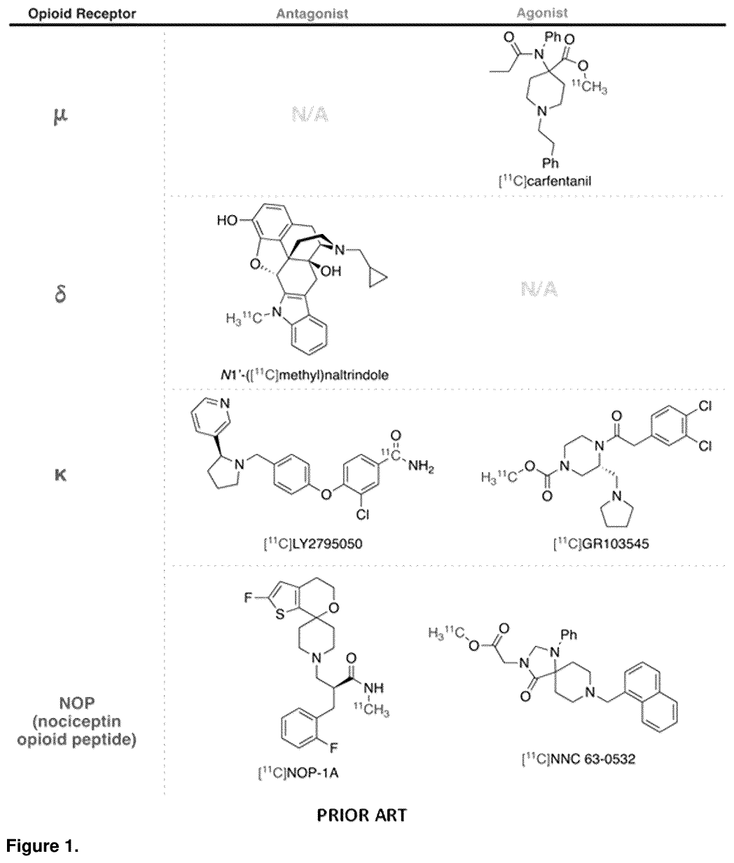

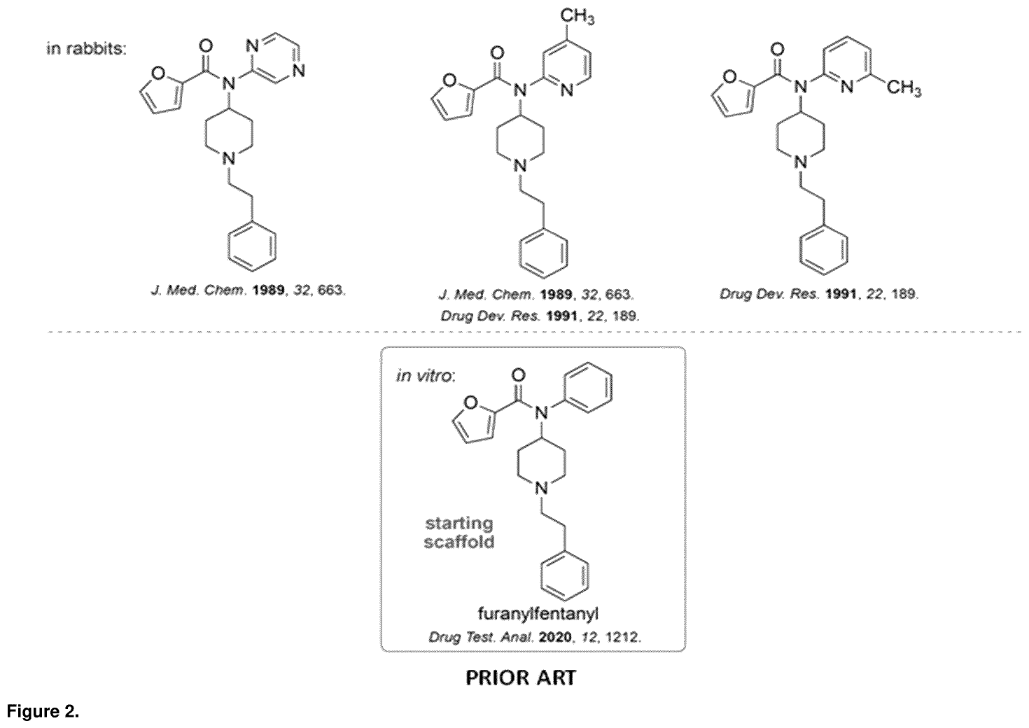

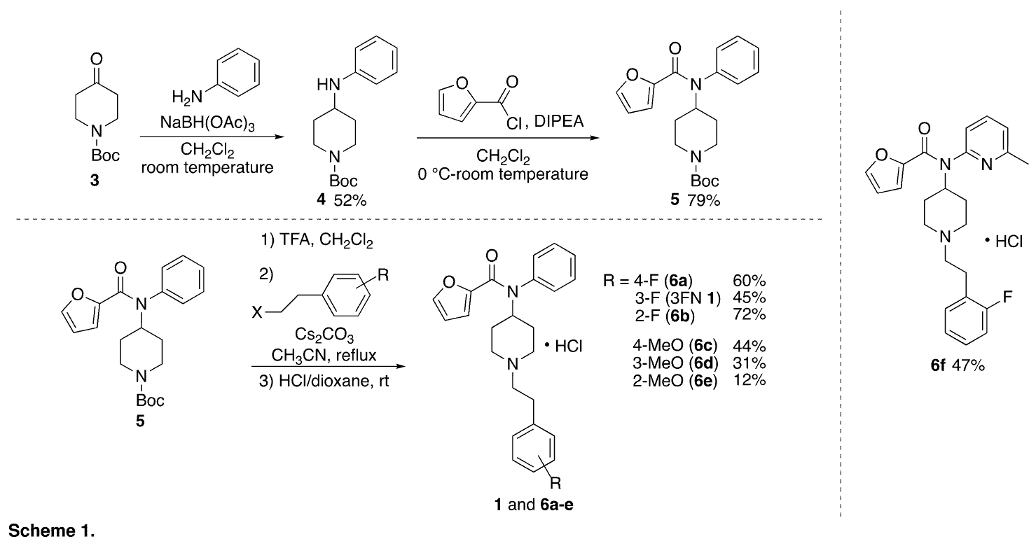

The compounds of the disclosure advantageously maintain good affinity for the desired receptor and can cross the blood-brain barrier. While there are reported agonist/antagonist radioligand pairs for other opioid receptors, only an agonist radioligand, [¹¹C] carfentanil is available for imaging the μ-opioid receptor, shown in FIG. 1. However, [¹¹C] carfentanil presents challenges regarding safety, and pharmacology. Carfentanil has estimated analgesic potency approximately 10,000 times that of morphine and 20-30 times that of fentanyl, [¹¹C] carfentanil is highly potent and presents issues with safety. Additionally, little is known about the pharmacology of carfentanil in humans. Its high potency and presumed high lipophilicity, large volume of distribution, and potential active metabolites have raised concerns about carfentanil administration and exposure. Therefore, a selective μ-opioid receptor antagonist can enable further studies of carfentanil as well. As shown in FIG. 2, several compounds are reported to exhibit antagonist properties and generally feature a furanyl group (2- or 3-substituted). These known compounds also exhibit some agonist activity as well as antagonist activity, which precludes their use as pure antagonists of the μ-opioid receptor. Moreover, the compounds known to exhibit antagonist properties all feature an unsubstituted phenylethylene moiety on the N atom of the piperazinyl ring. Compounds in accordance with the disclosure utilize furanylfentanyl as the starting scaffold, but as compared to prior known compounds, the compounds of the disclosure exhibit only antagonist activity. Additionally, it has been found that selection of the substitute substitution on the phenylethylene moiety resulted in compounds of the disclosure being capable of having the desired antagonist activity.

Compounds in accordance with the disclosure can have a structure of Formula (I):

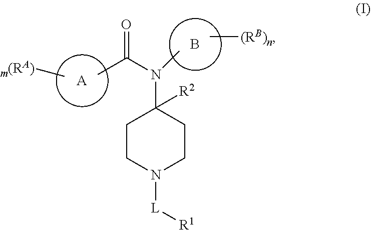

wherein the substituents are described in detail below.

Compounds in accordance with the disclosure can have a structure of Formula (II):

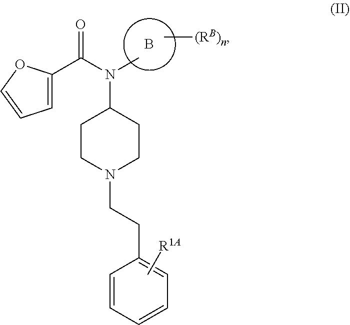

wherein the substituents are described in detail below.

The compounds described herein can be used to image the μ-opioid receptor. When the compounds are radio-labeled with, for example, ¹⁸F or ¹¹C, they can be useful for PET imaging, for example, and can result in improved diagnostic accuracy, image quality and shortening the procedure to one patient visit.

### μ-Opioid Receptor Antagonists of Formula (I)

Provided herein are compounds, or pharmaceutically acceptable salts thereof, having a structure of Formula (I):

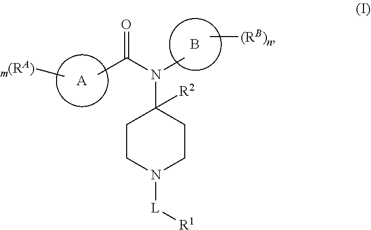

wherein:

each m and n are independently 0, 1, 2, or 3;

ring A is a 4-8 membered heterocycle, or 5-10 membered heteroaryl, wherein the heterocycle and heteroaryl each comprises 1, 2, or 3 ring heteroatoms selected from N, O, and S;

each R^{A}, when present, is C₁₋₃alkyl or C₁haloalkyl;

ring B is a C₆₋₁₀aryl, or 5-10 membered heteroaryl, wherein the heteroaryl comprises 1, 2, or 3 ring heteroatoms selected from N, O, and S;

each R^{B}, when present, is C₁₋₃alkyl or C₁haloalkyl;

L is C₂₋₆alkylene;

R¹ is halo, C₁₋₆alkoxyl, C₁₋₆haloalkyl, C₁₋₆haloalkoxyl, C₆₋₁₀aryl, 4-8 membered heterocycle, or 5-10 membered heteroaryl, wherein the heterocycle and heteroaryl each comprises 1, 2, or 3 ring heteroatoms selected from N, O, and S, and when R¹ is C₆₋₁₀aryl, 4-8 membered heterocycle or 5-10 membered heteroaryl, R¹ is substituted with 1 to 3 R^{1A} groups;

each R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X;

R² is H, —C(O)O—C₁₋₃alkyl, —C(O)O—C₁₋₃alkylene—X, or —C(O)O—C₁₋₃alkylene—LG;

X is a radioisotope; and

LG is a leaving group;

with the proviso that: only one X is present or X is absent.

In compounds of Formula (I), ring A can be a 4-8 membered heterocycle, or 5-10 membered heteroaryl, wherein the heterocycle and heteroaryl each comprises 1, 2, or 3 ring heteroatoms selected from N, O, and S. In various cases, ring A is 5-10 membered heteroaryl, and comprises 1, 2, or 3 ring heteroatoms selected from N, O, and S. In various cases, ring A is pyrrolyl, pyrazolidinyl, imidazolyl, furanyl, thiophenyl, oxazolyl, isoxazolyl, thiazolyl, and isothiazolyl. In various cases, ring A is furanyl.

In various cases, the compound has a structure of Formula (Ia):

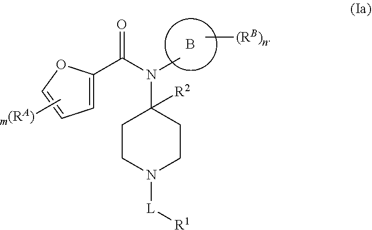

As disclosed herein, each of m and n are independently 0-3. In various cases, m is 0. In some cases, m is 1. In various cases, n is 0 or 1. In some cases, n is 1. In some cases, n is 0.

As disclosed herein, each R^{A} can independently be absent, C₁₋₃alkyl or C₁haloalkyl. In various cases, R^{A} is C₁alkyl or C₁haloalkyl. In various cases, R^{A} is C₁alkyl.

As disclosed herein, R¹ can be halo, C₁₋₆alkoxyl, C₁₋₆haloalkyl, C₁₋₆haloalkoxyl, C₆₋₁₀aryl, 4-8 membered heterocycle, or 5-10 membered heteroaryl, wherein the heterocycle and heteroaryl each comprises 1, 2, or 3 ring heteroatoms selected from N, O, and S, and when R¹ is C₆₋₁₀aryl, 4-8 membered heterocycle or 5-10 membered heteroaryl, R¹ is substituted with 1 to 3 R^{1A} groups. In various cases, R¹ is halo, C₁₋₆alkoxyl, C₁₋₆haloalkyl, or C₁₋₆haloalkoxyl. In various cases, R¹ is C₆₋₁₀aryl, 4-8 membered heterocycle or 5-10 membered heteroaryl and R¹ is substituted with 1 to 3 R^{1A} groups. In various cases, R¹ is C₆₋₁₀aryl.

In various cases, the compound has a structure of Formula (Ib):

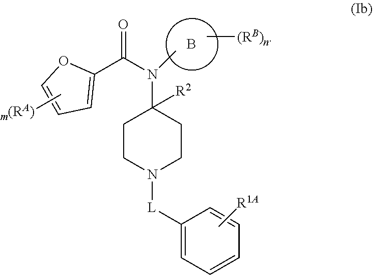

As disclosed herein, L can be C₂₋₆alkylene. In various cases, L is C₂₋₄alkylene. In various cases, L is C₂alkylene.

In various cases, the compound has a structure of Formula (Ic):

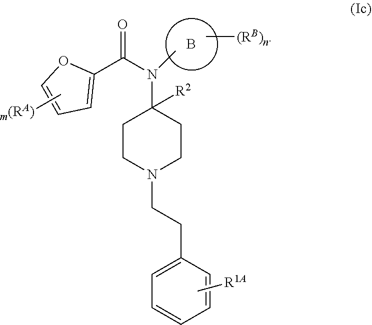

In various cases, the

compound has a structure of Formula (Id):

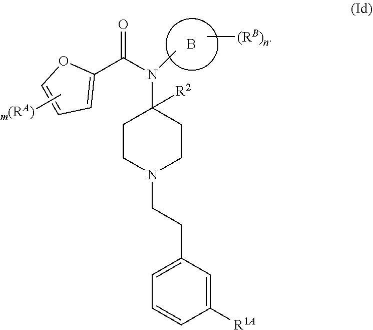

As disclosed herein, each R^{1A}, is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X. In various cases, R^{1A} is halo, C₁₋₃alkoxyl, C₁₋₃haloalkoxyl, X, or LG. In various cases, R^{1A} is C₁₋₃alkoxyl or C₁₋₃haloalkoxyl, and is substituted with X. In various cases, R^{1A} is X and the radioisotope is ¹⁸F. In various cases, R^{1A} is LG.

As disclosed herein, X can be a radioisotope. In various cases, X is ¹⁸F or ¹¹C. In various cases, X is ¹⁸F. In various cases, X is ¹¹C.

As disclosed herein, LG can be a leaving group. In various cases, LG is a hydroxyl, a trialkylstannane or a moiety containing I(III). In various cases, LG is a hydroxyl. In various cases, LG is SnMe₃ or SnBu₃. In various cases, LG is

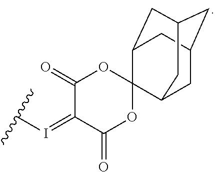 [SMILES: *I=C1C(=O)OC2(OC1=O)C1CC3CC(C1)CC2C3]

In compounds of the disclosure, when R^{1A} is LG, and LG is a hydroxyl, the H atom of the hydroxyl group is understood to be the leaving group.

As disclosed herein, ring B can be a C₆₋₁₀aryl, or 5-10 membered heteroaryl, wherein the heteroaryl comprises 1, 2, or 3 ring heteroatoms selected from N, O, and S. In various cases, ring B is a C₆₋₁₀aryl. In various cases, ring B is pyridyl.

In various cases, the compound has a structure of Formula (Ie):

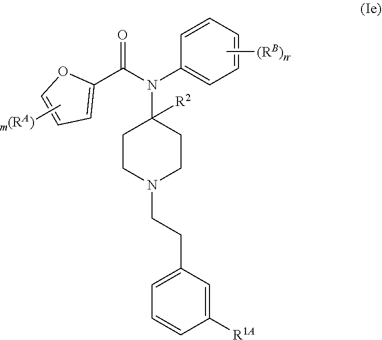

In various cases, the compound has a structure of Formula (If):

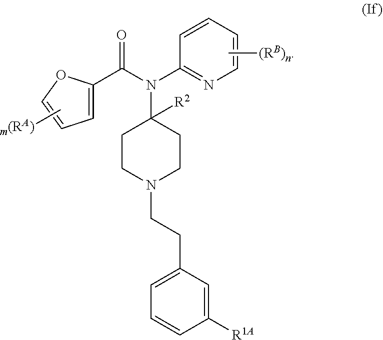

As disclosed herein, each R^{A} can independently be absent, C₁₋₃alkyl or C₁haloalkyl. In various cases, R^{A} is C₁alkyl or C₁haloalkyl. In various cases, R^{A} is C₁alkyl.

As disclosed herein, R² can be H, —C(O)O—C₁₋₃alkyl, —C(O)O—C₁₋₃alkylene—X, or —C(O)O—C₁₋₃alkylene-LG. In various cases, R² is —C(O)O—C₁₋₃alkyl, —C(O)O—C₁₋₃alkylene—X, or —C(O)O—C₁₋₃alkylene—LG. In various cases, R² is —C(O)O—C₁alkylene—X or —C(O)O—C₁₋₃alkylene—LG. In various cases, R² is —C(O)O—C₁alkylene—X and the radioisotope is ¹³C. In various cases, R² is —C(O)O—C₁₋₃alkylene—LG.

### μ-Opioid Receptor Antagonists of Formula (II)

Also provided herein are compounds, or pharmaceutically acceptable salts thereof, having a structure of Formula (II):

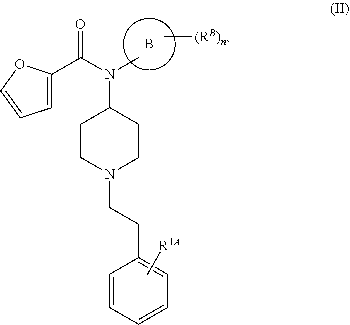

wherein:

n is 0 or 1;

ring B is phenyl or pyridyl;

each R^{B}, when present, is C₁alkyl or C₁haloalkyl;

R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X,

X is a radioisotope; and

LG is a leaving group

with the proviso that exclusively one X is present.

In various cases, the compound has a structure of Formula (IIa):

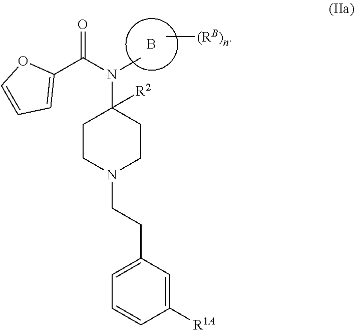

As disclosed herein, ring B is phenyl or pyridyl. In various cases, ring B is phenyl. In various cases, ring B is pyridyl.

In various cases, the compound has a structure of Formula (IIb):

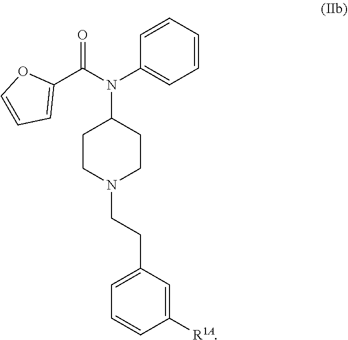

In various cases, the compound has a structure of Formula (IIc):

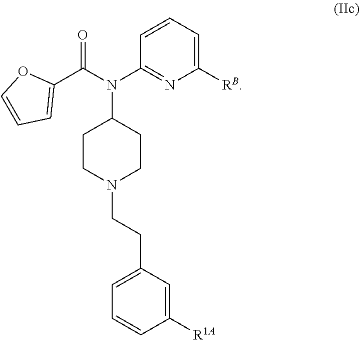

As disclosed herein, n can be 0 or 1. In various cases, n is 0. In various cases, n is 1.

As disclosed herein, R^{B} can be absent, C₁alkyl or C₁haloalkyl. In various cases, R^{B} is C₁alkyl.

As disclosed herein, R^{1A} can be halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X. In various cases, R^{1A} is halo, C₁₋₃alkoxyl, C₁₋₃haloalkoxyl, X, or LG. In various cases, R^{1A} is halo, X, or LG. In various cases, R^{1A} is C₁₋₃alkyl or C₁₋₃alkoxyl, R^{1A} is substituted with X, and the radioisotope is ¹¹C. In various cases, R^{1A} is C₁₋₃alkoxyl substituted with X, and the radioisotope is ¹⁸F. In various cases, R^{1A} is X and the radioisotope is ¹⁸F.

As disclosed herein, X can be a radioisotope. In various cases, X is ¹⁸F or ¹¹C. In various cases, X is ¹⁸F. In various cases, X is ¹¹C.

As disclosed herein, LG can be a leaving group. In various cases, LG is a hydroxyl, a trialkylstannane or a moiety containing I(III). In various cases, LG is a hydroxyl group. In various cases, LG is SnMe₃ or SnBu₃. In various cases, LG is

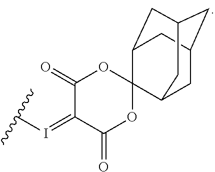 [SMILES: *I=C1C(=O)OC2(OC1=O)C1CC3CC(C1)CC2C3]

In compounds of the disclosure, when R^{1A} is LG, and LG is a hydroxyl, the H atom of the hydroxyl group is understood to be the leaving group.

Compounds as disclosed herein include those as shown in Tables A or B, or a pharmaceutically acceptable salt thereof.

TABLE A

| Ex | Structure |
|---|---|
| A1 | 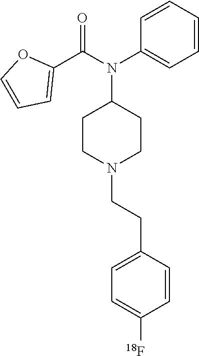 [SMILES: O=C(c1ccco1)N(c1ccccc1)C1CCN(CCc2ccc([18F])cc2)CC1] |
| A2 | 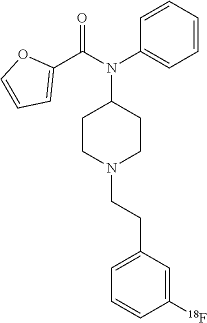 [SMILES: O=C(c1ccco1)N(c1ccccc1)C1CCN(CCc2cccc([18F])c2)CC1] |
| A3 | 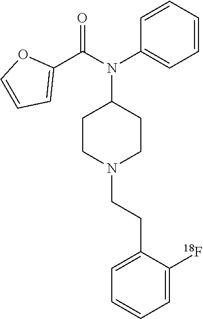 [SMILES: O=C(c1ccco1)N(c1ccccc1)C1CCN(CCc2ccccc2[18F])CC1] |
| A4 | 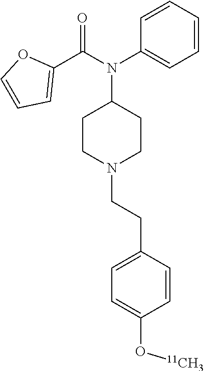 [SMILES: [11CH3]Oc1ccc(CCN2CCC(N(C(=O)c3ccco3)c3ccccc3)CC2)cc1] |
| A5 | 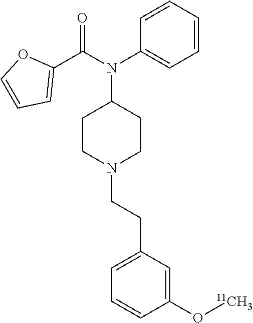 [SMILES: [11CH3]Oc1cccc(CCN2CCC(N(C(=O)c3ccco3)c3ccccc3)CC2)c1] |
| A6 | 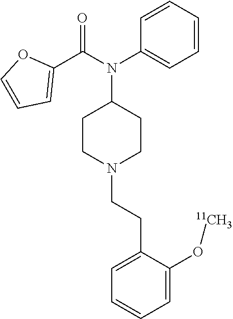 [SMILES: [11CH3]Oc1ccccc1CCN1CCC(N(C(=O)c2ccco2)c2ccccc2)CC1] |
| A7 | 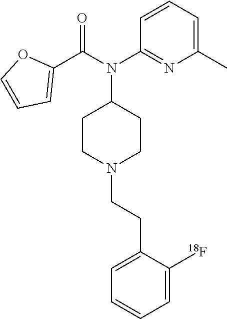 [SMILES: Cc1cccc(N(C(=O)c2ccco2)C2CCN(CCc3ccccc3[18F])CC2)n1] |

TABLE B

| Ex | Structure |
|---|---|
| B1 | 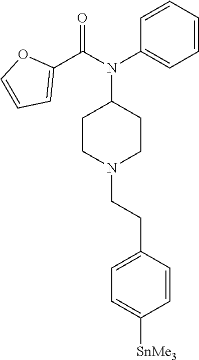 |
| B2 | 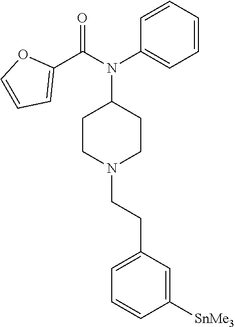 |
| B3 | 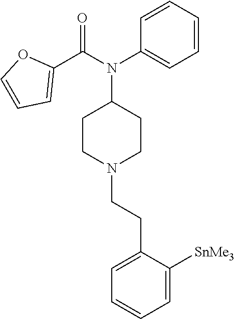 |
| B4 | 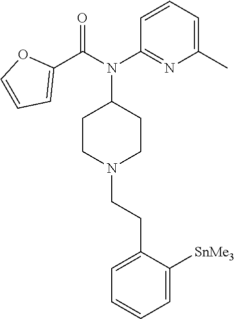 |
| B5 | 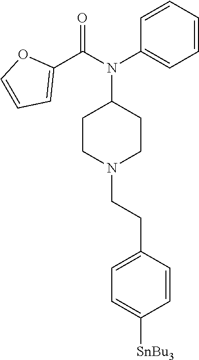 |
| B6 | 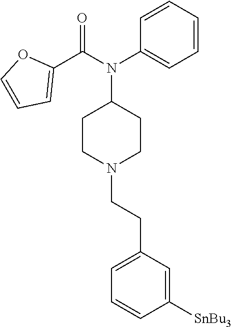 |
| B7 | 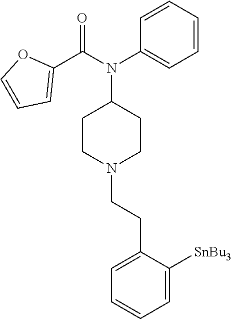 |
| B8 | 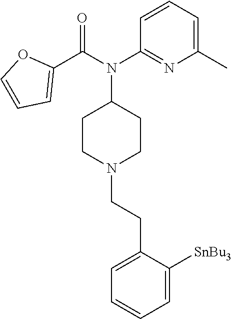 |
| B9 | 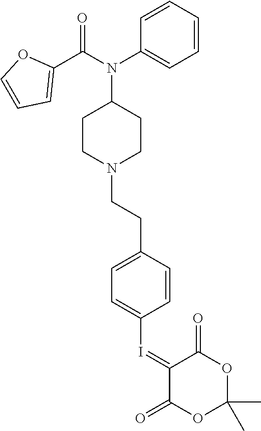 [SMILES: CC1(C)OC(=O)C(=Ic2ccc(CCN3CCC(N(C(=O)c4ccco4)c4ccccc4)CC3)cc2)C(=O)O1] |
| B10 | 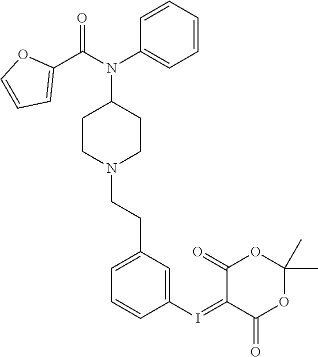 [SMILES: CC1(C)OC(=O)C(=Ic2cccc(CCN3CCC(N(C(=O)c4ccco4)c4ccccc4)CC3)c2)C(=O)O1] |
| B11 | 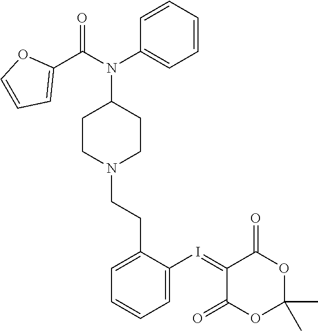 [SMILES: CC1(C)OC(=O)C(=Ic2ccccc2CCN2CCC(N(C(=O)c3ccco3)c3ccccc3)CC2)C(=O)O1] |
| B12 | 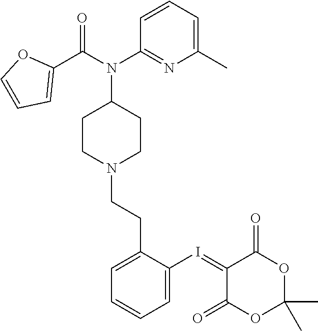 [SMILES: Cc1cccc(N(C(=O)c2ccco2)C2CCN(CCc3ccccc3I=C3C(=O)OC(C)(C)OC3=O)CC2)n1] |
| B13 | 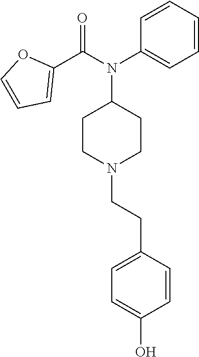 [SMILES: O=C(c1ccco1)N(c1ccccc1)C1CCN(CCc2ccc(O)cc2)CC1] |
| B14 | 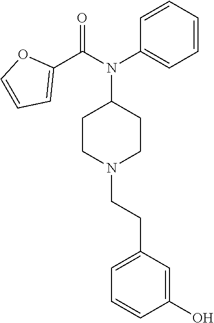 [SMILES: O=C(c1ccco1)N(c1ccccc1)C1CCN(CCc2cccc(O)c2)CC1] |
| B15 | 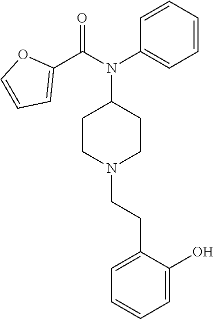 [SMILES: O=C(c1ccco1)N(c1ccccc1)C1CCN(CCc2ccccc2O)CC1] |

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

The compounds of Formulae (I) and (II) can have any stereochemical configuration at any sp³ carbon atoms. In some cases, the compounds of the disclosure are optically pure. As used herein, “optically pure” refers to the predominant presence of one enantiomer of a compound if multiple stereochemical configurations can exist (e.g., at least 99% enantiomeric excess). Unless otherwise indicated, all tautomeric forms of the compounds of the disclosure are within the scope of the disclosure.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

As used herein, the term “alkyl” refers to straight chained and branched saturated hydrocarbon groups. The term Cn means the alkyl group has “n” carbon atoms. For example, C₄ alkyl refers to an alkyl group that has 4 carbon atoms. C₁₋₆alkyl refers to an alkyl group having a number of carbon atoms encompassing the entire range (i.e., 1 to 6 carbon atoms), as well as all subgroups (e.g., 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “alkylene” refers to a bivalent saturated aliphatic radical. The term Cn means the alkylene group has “n” carbon atoms. For example, C₁₋₆alkylene refers to an alkylene group having a number of carbon atoms encompassing the entire range, as well as all subgroups, as previously described for “alkyl” groups.

As used herein, the term “aryl” refers to an aromatic carbocycle, and can be monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, fluorenyl, tetralinyl. Unless otherwise indicated, an aryl group can be an unsubstituted aryl group or a substituted aryl group.

As used herein, the term “heterocycle” refers to a non-aromatic ring which contains one to four heteroatoms independently selected from oxygen, nitrogen, and sulfur. Additionally, heterocycles of the disclosure can be monocyclic, bicyclic, bridged, fused or spirocyclic. For example, a heterocycle can be a monocyclic, bicyclic, bridged, fused, or spirocyclic 4-8 membered ring having 1 or 2 or 3 heteroatoms selected from N, O, and S. As another example, a heterocycle can be a 8-10 membered bicyclic, bridged, fused, or spirocyclic group having 1 or 2 or 3 ring heteroatoms selected from N, O, and S in the bicyclic ring. Nonlimiting examples of heterocycle groups include piperidine, piperazine, tetrahydrofuran, tetrahydropyran, dihydrofuran, morpholine, and oxazepane.

As used herein, the term “heteroaryl” refers to a cyclic aromatic ring having heteroatoms in the ring (e.g., a monocyclic aromatic ring with 5-6 total ring atoms, or a fused bicyclic ring with 9-10 total ring atoms), and containing one to three heteroatoms selected from nitrogen, oxygen, and sulfur atom in the aromatic ring. Unless otherwise indicated, a heteroaryl group can be unsubstituted or substituted. Heteroaryl groups can be isolated (e.g., pyridyl) or fused to another heteroaryl group (e.g., purinyl), a cycloalkyl group (e.g., tetrahydroquinolinyl), a heterocycle group (e.g., dihydronaphthyridinyl), and/or an aryl group (e.g., benzothiazolyl, quinolyl, isoquinolinyl, or quinazolinyl).

As used herein, the term “alkoxy” refers to a “—O-alkyl” group.

As used herein, the term “halogen” refers to fluorine, chlorine, bromine, and iodine. In some cases, the halo is a radioactive halogen. Examples of radioactive halogens include, but are not limited to, carbon-11, fluorine-18, chlorine-37, bromine-77, and iodine-124, iodine-131.

As used herein, the term “haloalkyl” refers to a “alkyl” group substituted with 1 or more halogens.

As used herein, the term “haloalkoxyl” refers to a “—O-alkyl” group substituted with 1 or more halogens.

As used herein, the term “leaving group” refers to any atom or moiety that is capable of being displaced by another atom or moiety in a chemical reaction. Examples of suitable leaving groups include, but are not limited to, trialkylstannanes, for example, SnMe₃ or SnBu₃: and groups that include I(III) moieties, for example,

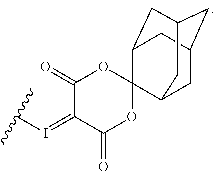 [SMILES: *I=C1C(=O)OC2(OC1=O)C1CC3CC(C1)CC2C3]

As used herein, a “substituted” functional group is a functional, group having at least one hydrogen radical that is substituted with a non-hydrogen radical (i.e., a substituent). Examples of non-hydrogen radicals (or substituents) include, but are not limited to, alkyl, ether, hydroxyl, alkoxyl, ester, acyl, carboxyl, amino, and halo. When a substituted alkyl group includes more than one non-hydrogen radical, the substituents can be bound to the same carbon or different carbon atoms.

### Pharmaceutically Acceptable Salts

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺ (C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g. enteral or parenteral). All methods that can be used by those skilled in the art to administer a pharmaceutically active agent are contemplated. In some cases, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Making Radiolabeled μ-Opioid Receptor Antagonists

The disclosure further provides methods of preparing radiolabeled μ-opioid receptor antagonists.

The disclosure provides a method including admixing a furanylfentanyl compound containing a leaving group and a radiolabeled source to form a compound having a structure of Formulae (I) or (II).

The radiolabeled source can include carbon-11 or fluorine-18. The fluorine-18 source is not particularly limited. In embodiments, the fluorine-18 source includes H-¹⁸F. Other suitable sources of fluorine-18 for use in the methods described herein include, but are not limited to fluorine-18 salts having counterions such as K, Na, Cs, or transition metals, such as Ag. For example, the fluorine-18 source can include K—¹⁸F, Na—¹⁸F, Cs—¹⁸F, or Ag—¹⁸F. The carbon-11 source is not particularly limited.

### Use of μ-Opioid Receptor Antagonists

The disclosure further provides methods of using the compounds described herein. In particular, the disclosure provides methods including administering to a subject a compound as described herein and subjecting the subject to an imaging modality.

The manner of administration of the compound is not particularly limited. For example, in embodiments, the compound can be administered intravenously or orally. The manner of administration and dose thereof would be within the purview of the doctor, nurse, or radiologist trained to administer these compounds.

In embodiments, the imaging modality can be selected from positron emission tomography (PET), positron emission tomography/computed tomography (PET/CT), positron emission tomography/magnetic resonance imaging (PET/MRI), planar gamma camera imaging, single-photon emission computerized tomography (SPECT), and/or single-photon emission computerized tomography/computed tomography (SPECT/CT).

Generally, it is envisaged that the compounds disclosed herein include a radioisotope when the subject is subjected to the imaging modality.

In embodiments, the subject is subjected to the imaging modality at a point in time ranging from about 0.5 hours to 7 days after of the compound. The time at which the subject is subjected to the imaging modality is dependent on the radioisotope used in the μ-opioid receptor antagonists. For example, due to the short half-life of ¹⁸F, when the compound is radiofluorinated, the subject can be subjected to the imaging modality at a point in time ranging from about 0.5 hours to about 5 hours, about 0.6 hours to about 4.5 hours, about 0.7 hours to about 4 hours, about 0.8 hours to about 3.5 hours, about 0.9 hours to about 3 hours, or about 1 hour to about 2 hours, for example at about 0.5, 0.6, 0.7, 0.8, 0.9, 1, 1.5, 2, 2.5, 3, 3.5, 4, 4.5, or 5 hours after administration of the compound.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

### Methods and Materials

All the chemicals were purchased from commercially available suppliers and used without purification. Unless otherwise stated, reagents and solvents were commercially available and used without further purification: sodium chloride, 0.9% USP, and sterile water for injection, USP, were purchased from Hospira; ethanol was purchased from American Regent; HPLC grade acetonitrile was purchased from Fisher Scientific. Other synthesis components were obtained as follows: sterile filters were obtained from Millipore; sterile product vials were purchased from Hollister-Stier; C18 Sep-Paks were purchased from Waters Corporation. C18 Sep-Paks were flushed with 10 mL of ethanol followed by 10 ml of water prior to use. Unless otherwise noted, all tissue culture reagents and radiolabeled ligands were purchased from commercial sources.

### Cell Lines and Membrane Preparations

Membranes prepared from transfected Chinese Hamster Ovary cells stably expressing human MOR, DOR, or KOR were used for all assays. Cells were grown to confluence at 37° C. in 5% CO₂ in Dulbecco's modified Eagle's medium (MOR, KOR) or Ham's F12 medium (DOR) containing 10% v/v fetal bovine serum (MOR, KOR) or 10% Fetal Clone II (DOR) and 5% v/v penicillin/streptomycin. Membranes were prepared from confluent cells by first detaching cells from the plates by incubation in warm harvesting buffer (20 mM HEPES, 150 mM NaCl, 0.68 mM EDTA, pH 7.4) followed by centrifugation at 200 g for 3 min. The cell pellet was suspended in ice-cold 50 mM Tris-HCl buffer, pH 7.4, and homogenized with a Tissue Tearor (Biospec Products, Inc.) for 20 s at setting 4. The homogenate was centrifuged at 20000 g for 20 min at 4° C., and the pellet was rehomogenized in 50 mM Tris-HCl, pH 7.4, with a Tissue Tearor for 10 s at setting 2, followed by recentrifugation. The final pellet was resuspended in 50 mM Tris-HCl, pH 7.4 and frozen in aliquots at −80° C. Protein concentration was determined via Pierce BCA protein assay kit using bovine serum albumin as the standard.

### Binding Affinity

Binding affinities for fentanyl derivatives 1 and 6a-e at KOR, MOR, and DOR were determined by competitive displacement of [³H] diprenorphine as previously reported. In a 96-well plate, cell membranes (10-20 μg of protein) and [³H] diprenorphine (0.2 nM) were shaken in Tris-HCl buffer (50 mM, pH 7.4) with various concentrations of test compound at room temperature for 1 h, allowing the mixture to reach equilibrium. Nonspecific binding was determined using the opioid antagonist naloxone (10 μM), and total binding was determined using vehicle in the absence of competitive ligand. After incubation, membranes were filtered through Whatman GF/C 1.2 μm glass fiber filters and washed with 50 mM Tris-HCl buffer. The radioactivity remaining on the filters was then quantified by liquid scintillation counting in a PerkinElmer Microbeta 2450 after saturation with EcoLume liquid scintillation cocktail. Binding affinity (Kᵢ) values were calculated via “One-site-Fit Ki” nonlinear regression analysis using GraphPad Prism software from at least three independent binding assays performed in duplicate.

### Stimulation of [³⁵S]GTPγS Binding

Agonist stimulation of KOR, MOR, and DOR by fentanyl derivatives 1 and 6a-e was determined by [³⁵S]guanosine 5′-O-[γ-thio]triphosphate ([³⁵S]-GTPγS) binding assays as previously reported. In a 96-well plate, membranes from cells expressing opioid receptors as described above (10-20 μg of protein), [³⁵S] GTPγS (0.1 nM), and guanosine diphosphate (30 μM) were incubated with gentle agitation in GTPγS buffer (50 mM Tris-HCl, 100 mM NaCl, 5 mM MgCl₂, 1 mM EDTA, pH 7.4) with various concentrations of test compound at 25° C. for 1 h. Basal stimulation was determined by incubation in the absence of any ligand. After incubation, membranes were filtered through Whatman GF/C 1.2 μm glass fiber filters and washed with GTPγS buffer with no EDTA. The radioactivity remaining on the filters was then quantified by liquid scintillation counting in a PerkinElmer Microbeta 2450 after saturation with EcoLume liquid scintillation cocktail. Data are reported as percent stimulation compared to the effects of 10 μM standard agonist: U69,593 (KOR), DAMGO (MOR), or SNC80 (DOR). Percent stimulation and EC50 values were determined via sigmoidal dose-response nonlinear regression analysis using GraphPad Prism software from at least three independent assays performed in duplicate. Efficacy is expressed as percent stimulation relative to 10 μM standard agonist.

### Instrumental Information

Automated flash chromatography was performed with Biotage Isolera Prime system. High-performance liquid chromatography (HPLC) was performed using a Shimadzu LC-2010A HT system equipped with a Bioscan B-FC-1000 radiation detector. ¹H and ¹³C NMR spectra: Varian 500 apparatus (400 MHz for ¹H NMR and 100 MHz for ¹³C NMR), in DMSO-d₆ or CDCl₃ unless otherwise indicated, δ in ppm relative to tetramethylsilane (δ=0), J in Hz. Mass spectrometry (HRMS) was performed using an Agilent 6520 Accurate-Mass Q-TOF LC/MS spectrometer using ESI ionization with less than 5-ppm error for all HRMS analyses.

### Example 1—Synthesis of Radiochemical Precursor Compounds of the Disclosure

The lack of a handle that could be readily converted to its' corresponding ¹¹C or ¹⁸F radioisotope directed compound design towards substituted analogs of furanylfentanyl. Analogs were made that contained methoxy or fluoro substituents on the phenyl group of the phenethyl appendage. Commercially available tert-butyl 4-oxopiperidine-1-carboxylate 3 was treated with aniline and NaBH(OAc)₃ in dichloromethane at room temperature time for 48 h to give secondary amine 4 which was then acylated with 2-furoyl chloride in dichloromethane with DIPEA as the base after stirring for 72 h at room temperature, resulting in common intermediate 5 (Scheme 1).

Acylated amine 5 was then Boc deprotected by treatment with TFA in dichloromethane at room temperature for 3 h and then alkylated with the desired phenethyl halide as a crude residue by refluxing overnight with Cs₂CO₃ in acetonitrile to afford the desired fentanyl analogs that were converted into HCI salts 3FN 1 and 6a-e using 4 M HCl/1,4-dioxane. An additional pyridine containing compound 6f was synthesized using the same synthetic sequence reported above (instead of aniline, 6-methylpyridin-2-amine was used in the reductive amination step).

### Synthesis of Boc Protected Core (5)

The synthesis of Boc amine compound 1 was performed according to Scheme 2. To a round bottom flask was added tert-butyl 4-oxopiperidine-1-carboxylate (1 mmol) followed by 3.33 mL of dichloromethane. The desired aniline derived compound (1.1 mmol) was added then acetic acid (0.99 mmol). The reaction mixture was then cooled to 0° C. and NaBH(OAc)₃ (0.99 mmol) was added slowly portionwise. The mixture was allowed to warm up to room temperature and stirred for 24 h. An additional batch of NaBH(OAc)₃ was then added portionwise and the mixture was stirred for another 24 h. After pouring into a sat aq. NaHCO₃ solution and separating the organic layer, the aqueous layer was extracted 2× with 5 mL of dichloromethane. The combined organic layers were then dried over Na₂SO₄ and concentrated in vacuo. The residue was then purified by column chromatography (1-5% methanol/dichloromethane) to afford the desired Boc amine compound 1.

The synthesis of Boc amide compound 2 was performed according to Scheme 3. A solution of Boc amine 1 (1 mmol)/dichloromethane (10 mL) in a round bottom flask was cooled to 0° C. and to it was added diisopropylethylamine (2.0 mmol). Acid chloride was then added slowly dropwise to the reaction mixture and stirred for 72 h. The mixture was then diluted with water (10 mL), washed with an aqueous solution of saturated NaHCO₃ and brine. The organic layer was then separated, dried over Na₂SO₄ and concentrated in vacuo. Purification of the resulting residue by column chromatography (15-70% ethyl acetate/hexanes) gave the desired Boc amide 2.

The synthesis of secondary amine 4 was performed according to Scheme 4. Commercially available tert-butyl 4-oxopiperidine-1-carboxylate 3 (3.0 g, 15.1 mmol) was dissolved with dichloromethane (50 mL) in a round bottom flask at room temperature. Aniline (1.52 mL, 16.6 mmol) was added followed by dropwise addition of acetic acid (852 L, 14.9 mmol). The reaction mixture was cooled to 0° C. followed by slow batchwise addition of NaBH(OAc)₃ (3.16 g, 14.9 mmol). Upon warming to room temperature the reaction was stirred for 24 h followed by slow batchwise addition of another portion of NaBH (OAc)₃ (3.16 g, 14.9 mmol). After an additional 24 h, the mixture was poured slowly into a saturated aqueous sodium bicarbonate solution (50 mL). The organic layer was separated and the aqueous layer was extracted with dichloromethane (2×50 mL). The combine organic layers were then dried over Na₂SO₄ and concentrated in vacuo. The resulting residue was purified by flash column chromatography (1-5% methanol/dichloromethane) to give secondary amine 4 as a light tan solid (2.15 g, 52% yield).

A: To a Boc amide 2 (1 mmol) solution in dichloromethane (2.77 mL) was added TFA (1.39 mL) slowly dropwise. The reaction was allowed to stir for 3 hr, cooled to 0° C. and quenched with 1 M NaOH. The organic layer was then washed with 1 M NaOH, saturated aqueous NaHCO3 and brine. After drying over Na2SO4 the solvent was evaporated in vacuo and used directly for the next step.

B: The crude residue from step 3A was dissolved in acetonitrile (10 mL) and then Cs2CO3 followed by the desired alkyl halide were added to a round bottom flask. The reaction mixture was then heated at reflux for 16 h. After cooling to room temperature, the mixture was extracted 3× with dichloromethane (3×10 mL). The combined organic layers were washed with saturated aqueous NaHCO₃ and brine. After drying over Na₂SO₄ the solvent was removed in vacuo and purified by column chromatography (1-10% methanol/dichloromethane) to afford the desired fentanyl analogs 3.

C: Desired fentanyl analog 3 (1 mmol) was dissolved in 6 N HCl/dioxane (10 mL) and allowed to stir at room temperature for 16 h. The volatiles were then removed in vacuo and the residue was triturated using dichloromethane/hexanes. The resulting solid was filtered and dried to give the desired fentanyl analog HCl salt 4.

To a round bottom flask containing compound 4 (1.15 g, 4.16 mmol) in dichloromethane (42 mL) at 0° C. was added Hunig's base (1.45 mL, 8.32 mmol) slowly dropwise. 2-Furoyl chloride (817 μL, 8.32 mmol) was then added slowly dropwise and the reaction mixture was warmed to room temperature. After 72 h, the reaction mixture was diluted with H₂O (42 mL), washed with saturated NaHCO₃, brine, dried over Na₂SO₄ and concentrated in vacuo. The crude residue was purified by flash column chromatography (15-70% ethyl acetate/hexanes) to afford the desired acylated amine 5 as a light yellow solid (1.22 g, 79% yield).

tert-Butyl 4-(phenylamino)piperidine-1-carboxylate (4). The desired product was obtained as a light tan solid. ¹H NMR (400 MHZ, CDCl₃) 0δ 7.21-7.14 (m, 2H), 6.76-6.69 (m, 1H), 6.67-6.60 (m, 2H), 4.05 (bs, 1H), 3.47-3.37 (m, 1H), 2.91 (t, J=12 Hz, 2H), 2.09-1.98 (m, 2H), 1.46 (s, 9H), 1.40-1.27 (m, 2H); ¹³C NMR (100 MHz, CDCl₃) δ 154.8, 150.4, 146.2, 129.4, 118.0, 113.7, 79.6, 50.5, 32.2, 28.4.

tert-Butyl 4-(N-phenylfuran-2-carboxamido)piperidine-1-carboxylate (5). Compound 5 was isolated as a light yellow solid. ¹H NMR (400 MHZ, CDCl₃) δ 7.47-7.36 (m, 3H), 7.31 (s, 1H), 7.17-7.05 (m, 2H), 6.14-6.09 (m, 1H), 5.42-5.35 (m, 1H), 4.95-4.84 (m, 1H), 4.14 (d, J=16 Hz, 2H), 2.84 (t, J=14 Hz, 2H), 1.90-1.80 (m, 2H), 1.42-1.36 (m, 9H), 1.36-1.26 (m, 2H); ¹³C NMR (100 MHZ, CDCl₃) δ 158.8, 154.6, 147.0, 144.5, 138.4, 130.8, 129.3, 128.9, 116.1, 111.0, 79.6, 53.2, 43.2, 30.2, 28.4.

### General Procedure for Alkylation and Generation of Fentanyl Derivative Salts 1 and 6a-e

Boc protected amine 5 (100 mg, 0.27 mmol) was dissolved in dichloromethane (740 μL) at room temperature. TFA (370 μL) was then slowly added dropwise and the resulting solution was allowed to stir for 3 h. After cooling to 0° C., the reaction was quenched with 1 M NaOH (2 mL), washed with saturated NaHCO₃ and brine. The organic layer was dried over Na₂SO₄ and concentrated in vacuo. The crude secondary amine was used without purification for the next step. Crude secondary amine (0.1 mmol) was dissolved in acetonitrile (1 mL) at room temperature and Cs₂CO₃ (0.2 mmol) was added. The desired phenethyl halide (0.105 mmol) was added dropwise and the reaction mixture was heated to reflux. After 24 h, the mixture was cooled to room temperature, quenched with H₂O (1 mL) and extracted with dichloromethane (3×1 mL). The combined organic layers were washed with saturated NaHCO₃, brine, dried over Na₂SO₄ and concentrated in vacuo. The crude residue was purified by flash column chromatography (1-10% methanol/dichloromethane) to afford the free base fentanyl derivative. The free base was then dissolved in 4 M HCl/1,4-dioxane (5 mL) and stirred overnight. After concentration in vacuo the residue was triturated with dichloromethane/hexanes and filtered to give the desired HCl salts 1 and 6a-e.

N-(1-(3-Fluorophenethyl)piperidin-4-yl)-N-phenylfuran-2-carboxamide HCl (3FN (1)). The desired product 1 was afforded as an off-white solid (37.5 mg, 45% yield). ¹H NMR (400 MHZ, DMSO-d6) δ 10.09 (bs, 1H), 7.66 (s, 1H), 7.56-7.47 (m, 3H), 7.42-7.28 (m, 3H), 7.18-7.04 (m, 3H), 6.33 (s, 1H), 5.44 (s, 1H), 4.91-4.77 (m, 1H), 3.65-3.51 (m, 2H), 3.28-3.10 (m, 4H), 3.07-2.96 (m, 2H), 2.07 (d, J=8 Hz, 2H), 1.81-1.67 (m, 2H); ¹³C NMR (100 MHZ, DMSO-d6) δ 163.7, 161.7, 158.5, 147.0, 145.7, 140.4, 138.3, 131.3, 131.1, 131.0, 125.3, 116.3, 116.0, 115.9, 114.2, 114.0, 111.7, 66.8, 56.5, 51.1, 29.5, 27.5; ¹⁹F NMR (376 MHz, DMSO-d6) δ-113.0-(-) 113.3 (m, 1F); HRMS (ESI⁺-TOF) (C₂₄H₂₆FN₂O₂) calcd 393.1900 (M+H), found 393.2065.

N-(1-(4-Fluorophenethyl)piperidin-4-yl)-N-phenylfuran-2-carboxamide HCl (6a). Compound 6a was obtained as a white solid (41.2 mg, 60% yield). ¹H NMR (400 MHZ, DMSO-d6) δ 10.1 (bs, 1H), 7.64 (s, 1H), 7.56-7.44 (m, 3H), 7.35-7.23 (m, 4H), 7.20-7.09 (m, 2H), 6.35-6.26 (m, 1H), 5.45-5.34 (m, 1H), 4.89-4.74 (m, 1H), 3.63-3.49 (m, 2H), 3.23-3.09 (m, 4H), 3.01-2.88 (m, 2H), 2.05 (d, J=12 Hz, 2H), 1.80-1.64 (m, 2H); ¹³C NMR (100 MHz, DMSO-d6) δ 160.4, 158.4, 146.9, 145.7, 138.3, 133.6, 131.2, 131.1, 131.0, 129.9, 129.5, 116.3, 116.0, 115.7, 111.7, 56.9, 51.4, 50.5, 29.0, 27.4; ¹⁹F NMR (376 MHZ, DMSO-d6) δ-116.0-(-) 116.2 (m, 1F).

N-(1-(2-Fluorophenethyl)piperidin-4-yl)-N-phenylfuran-2-carboxamide HCl (6b). The desired product 6b was obtained as an off-white solid (48.3 mg, 72% yield). ¹H NMR (400 MHZ, DMSO-d6) δ 10.3 (bs, 1H), 7.66 (s, 1H), 7.57-7.45 (m, 3H), 7.38-7.26 (m, 4H), 7.19 (q, J=6.7 Hz, 2H), 6.37-6.28 (m, 1H), 5.48-5.38 (m, 1H), 4.89-4.77 (m, 1H), 3.59 (d, J=8 Hz, 2H), 3.27-3.13 (m, 4H), 3.10-2.99 (m, 2H), 2.07 (d, J=12 Hz, 2H), 1.83-1.67 (m, 2H); ¹³C NMR (100 MHZ, DMSO-d6) δ 161.8, 159.9, 158.5, 147.0, 145.7, 138.3, 131.5, 131.2, 129.9, 129.7, 129.6, 125.2, 124.2, 124.1, 116.3, 116.0, 115.8, 111.7, 55.4, 51.4, 50.6, 27.5, 23.5; ¹⁹F NMR (376 MHZ, DMSO-d6) δ-118.1-(-) 118.4 (m, 1H).

N-(1-(4-Methoxyphenethyl)piperidin-4-yl)-N-phenylfuran-2-carboxamide HCl (6c). Compound 6c was obtained as an off-white solid (21.6 mg, 44% yield). ¹H NMR (400 MHz, DMSO-d6) δ 10.0 (bs, 1H), 7.67-7.62 (m, 1H), 7.53-7.46 (m, 3H), 7.34-7.25 (m, 2H), 7.17-7.10 (m, 2H), 6.89-6.84 (m, 2H), 6.31 (dd, J=3.6, 1.7 Hz, 1H), 5.46-5.35 (m, 1H), 4.81 (t, J=12.3 Hz, 1H), 3.70 (s, 3H), 3.60-3.48 (m, 2H), 3.22-3.04 (m, 4H), 2.94-2.84 (m, 2H), 2.04 (d, J=13.2 Hz, 2H), 1.71 (q, J=12.6 Hz, 2H); ¹³C NMR (100 MHZ, DMSO-d6) δ 158.6, 158.4, 146.9, 145.7, 138.3, 131.3, 130.2, 129.9, 129.5, 129.2, 116.3, 114.5, 111.7, 57.2, 55.5, 51.4, 50.6, 29.1, 27.4.

N-(1-(3-Methoxyphenethyl)piperidin-4-yl)-N-phenylfuran-2-carboxamide HCl (6d). The desired product 6d was afforded as an off-white solid (16.1 mg, 31% yield). ¹H NMR (400 MHZ, DMSO-d6) δ 10.0 (bs, 1H), 7.64 (s, 1H), 7.56-7.44 (m, 3H), 7.34-7.19 (m, 3H), 6.85-6.74 (m, 3H), 6.31 (s, 1H), 5.41 (s, 1H), 4.86-4.75 (m, 1H), 3.72 (s, 3H), 3.55 (d, J =8 Hz, 2H), 3.23-3.08 (m, 4H), 2.99-2.87 (m, 2H), 2.05 (d, J=12 Hz, 2H), 1.80-1.63 (m, 2H); ¹³C NMR (100 MHZ, DMSO-d6) δ 159.9, 158.4, 147.0, 145.6, 139.0, 138.3, 131.2, 130.2, 129.9, 129.5, 121.3, 116.3, 114.8, 112.7, 111.7, 56.8, 55.5, 51.4, 50.6, 29.9, 27.4.

N-(1-(2-Methoxyphenethyl)piperidin-4-yl)-N-phenylfuran-2-carboxamide HCl (6e). Compound 6e was obtained as an off-white solid (10.5 mg, 12% yield). ¹H NMR (400 MHz, DMSO-d6) δ 9.49 (bs, 1H), 7.64 (s, 1H), 7.54-7.46 (m, 3H), 7.35-7.28 (m, 2H), 7.24 (t, J=6 Hz, 1H), 7.15 (d, J=8 Hz, 1H), 6.98 (d, J=4 Hz, 1H), 6.89 (t, J=6 Hz, 1H), 6.32 (s, 1H), 5.44 (s, 1H), 4.87-4.77 (m, 1H), 3.77 (s, 3H), 3.57 (d, J=8 Hz, 2H), 3.22-3.07 (m, 4H), 2.93-2.85 (m, 2H), 2.06 (d, J=12 Hz, 2H), 1.75-1.62 (m, 2H); ¹³C NMR (100 MHZ, DMSO-d6) δ 158.4, 157.5, 147.0, 145.7, 138.4, 131.3, 130.5, 129.9, 129.5, 129.0, 125.0, 121.0, 116.3, 111.7, 111.4, 55.9, 55.7, 51.4, 50.5, 27.5, 25.1.

tert-butyl 4-(N-(6-methylpyridin-2-yl)furan-2-carboxamido) piperidine-1-carboxylate (5n). Compound 5n was obtained as an off-white solid (35.8 mg, 32% yield). ¹H NMR (400 MHZ, CDCl₃) δ 7.54 (t, J=8 Hz, 1H), 7.23-7.13 (m, 2H), 6.84 (d, J=8 Hz, 1H), 6.19 (dd, J=3.6, 1.7 Hz, 1H), 6.01-5.96 (m, 1H), 4.90-4.77 (m, 1H), 4.25-4.00 (m, 2H), 2.91-2.70 (m, 2H), 2.57 (s, 3H), 1.91 (d, J=12 Hz, 2H), 1.57-1.44 (m, 2H), 1.41 (s, 9H); ¹³C NMR (100 MHZ, CDCl₃) δ 158.8, 158.7, 154.7, 152.2, 147.5, 144.3, 138.2, 123.0, 121.8, 116.2, 111.0, 79.5, 54.0, 43.4, 30.2, 28.4, 24.3.

N-(1-(2-fluorophenethyl)piperidin-4-yl)-N-(6-methylpyridin-2-yl) furan-2-carboxamide (6f). The desired compound 6f was afforded as a white solid (39.9 mg, 47% yield). ¹H NMR (400 MHZ, DMSO-d6) δ 10.5 (bs, 1H), 7.80 (t, J=8 Hz, 1H), 7.63-7.56 (m, 1H), 7.40-7.27 (m, 3H), 7.23-7.10 (m, 3H), 6.42-6.32 (m, 1H), 5.90 (d, J=4 Hz, 1H), 4.83-4.71 (m 1H), 3.64-3.50 (m, 2H), 3.25-2.98 (m, 6H), 2.48 (s, 3H), 2.06-1.85 (m, 4H); ¹³C NMR (100 MHZ, DMSO-d6) δ 159.7, 158.3, 151.7, 147.3, 145.7, 139.5, 131.5, 125.2, 124.2, 124.1, 123.8, 122.3, 116.4, 116.0, 115.8, 111.8, 55.4, 51.4, 51.2, 27.4, 24.3, 23.4; ¹⁹F NMR (376 MHz, DMSO-d6) δ-118.2-(-) 118.4.

### Synthesis of Side Chain

1-Iodo-3-(2-iodoethyl)benzene (8). To a flame dried round bottom flask at room temperature was added methyl 2-(3-iodophenyl)acetate (2.5 g, 9.06 mmol) followed by THF (91 mL). The solution was then cooled to 0° C. and LiBH₄ (559 mg, 18.1 mmol) was added slowly batchwise. The reaction was warmed to room temperature and after 24 h was cooled back down to 0° C. The cooled mixture was quenched with H₂O (91 mL), extracted 3× with ethyl acetate (91 mL), dried over Na₂SO₄ and concentrated in vacuo. The crude residue was obtained as a clear oil and used directly in the next step (1.44 g, 64% yield). Previously obtained crude alcohol (1.44 g, 5.81 mmol) was dissolved in THF (19.4 mL) in a round bottom flask and cooled to 0° C. PPh₃ (1.52 g, 5.81 mmol) was then added followed by imidazole (554 mg, 8.13 mmol). I₂ (2.06 g, 8.13 mmol) was then added batchwise, the reaction mixture was warmed up to room temperature and allowed to stir for 72 h. The reaction mixture was quenched by pouring into ice water (20 mL), extracted with diethyl ether 3× (20 mL), washed with 10% by wt. sodium thiosulfate, dried over Na₂SO₄ and concentrated in vacuo. The crude residue was purified by flash column chromatography (1-30% ethyl acetate/hexanes) to afforded the desired iodo product 8 as a yellow oil (1.13 g, 54% yield). ¹H NMR (400 MHZ, CDCl₃) δ 7.63-7.58 (m, 1H), 7.57-7.53 (m, 1H), 7.19-7.13 (m, 1H), 7.06 (t, J=6 Hz, 1H), 3.32 (t, J=8 Hz, 2H), 3.12 (t, J=8 Hz, 2H); ¹³C NMR (100 MHz, CDCl₃) δ 142.8, 137.4, 136.0, 130.4, 127.7, 94.6, 39.6, 4.7.

Synthesis of N-(1-(3-iodophenethyl)piperidin-4-yl)-N-phenylfuran-2-carboxamide (7). General synthesis for fentanyl derivatives reported above was followed, using compound 8 as phenethyl halide as shown in Scheme 5. ¹H NMR (400 MHZ, CDCl₃) δ 7.54-7.48 (m, 2H), 7.45-7.38 (m, 3H), 7.35-7.31 (m, 1H), 7.20-7.10 (m, 3H), 6.99 (t, J=8 Hz, 1H), 6.13 (dd, J=3.6, 1.7 Hz, 1H), 5.38 (d, J=3.6 Hz, 1H), 4.88-4.76 (m, 1H), 3.18-2.96 (m, 2H), 2.78-2.46 (m, 4H), 2.40-2.19 (m, 2H), 1.92 (d, J=12 Hz, 2H), 1.73-1.47 (m, 2H); ¹³C NMR (100 MHZ, CDCl₃) δ 158.9, 147.1, 144.4, 138.4, 137.6, 135.3, 130.9, 130.2, 129.3, 128.9, 128.0, 116.1, 110.9, 110.8, 94.4, 59.9, 53.6, 52.9, 33.1, 30.0.

### Synthesis of Radiochemical Precursor

N-phenyl-N-(1-(3-(trimethylstannyl)phenethyl)piperidin-4-yl) furan-2-carboxamide (3-Me₃SnFN (2)). The synthesis of 3-Me₃SnFN (2) was performed as shown in Scheme 6. To a flame dried round bottom flask under a pillow of argon was added LiCl (39.3 mg, 0.926 mmol), compound 7 (96.6 mg, 0.193 mmol) and Pd(PPh₃)₄ (45.1 mg, 0.039 mmol) followed by vacuum and backfilling with argon (3×). Toluene (1.93 mL) was added and hexamethylditin (328 mg, 1 mmol) was added dropwise. The reaction mixture was heated to 100° C., stirred for 16 h and then cooled to room temperature. To the mixture was then added, 1 mL of aqueous 2 M KF and after stirring for an additional 30 mins the contents were filtered over celite. The filtrate was washed with brine, dried over Na₂SO₄ and concentrated in vacuo. The crude residue was purified by flash column chromatography (1-10% methanol/dichloromethane) to give stannane 2 as a brown oil (90.1. mg, 87% yield). Mixture of rotamers: ¹H NMR (400 MHZ, CDCl₃) δ 7.70-7.62 (m, 5H), 7.57-7.50 (m, 2H), 7.49-7.42 (m, 5H), 7.41-7.38 (m, 3H), 7.34-7.21 (m, 3H), 7.18-7.14 (m, 2H), 6.14 (m, 1H), 5.40-5.34 (m, 1H), 4.88-4.77 (m, 1H), 3.10-3.01 (m, 2H), 2.79-2.69 (m, 2H), 2.62-2.53 (m, 2H), 2.30-2.19 (m, 2H), 1.95-1.86 (m, 2H), 1.65-1.50 (m, 2H), 0.33-0.18 (m, 9H); ¹³C NMR (100 MHZ, CDCl₃) δ 158.9, 147.1, 144.33, 144.31, 142.4, 138.4, 136.2, 133.6, 133.0, 132.1, 132.03, 131.97, 131.9, 130.9, 129.3, 128.8, 128.6, 128.5, 128.1, 116.0, 110.9, 60.5, 53.1, 53.0, 33.8, 30.1, −9.6; HRMS (ESI⁺-TOF) (C₂₇H₃₅N₂O₂Sn) calcd 539.1642 (M+H), found 539.1767.

Purified precursor 2 was then aliquoted in ˜5 mg portions into amber glass vials with crimp-top septa caps to be easily used for test radiochemistry and full automation runs.

### Example 2—Radiolabeled Compounds of the Disclosure

A TRACERLab FXFN synthesis module was used for the automated radiosynthesis of [18F]3FN 1 (Scheme 7).

[¹⁸F]FluoFAPI 1 radiosynthesis was automated in a TRACERLab FXFN module. Briefly, cyclotron produced ¹⁸F was trapped on a QMA Sep-Pak and eluted into the reactor using KOTf (10 mg) and minimal K₂CO₃ (50 μg) in H₂O (0.5 mL). Azeotropic drying was then carried out using acetonitrile (1 mL) at 100° C. first under vacuum for 5 min then under vacuum with argon over pressure for an additional 5 min. To the dried [¹⁸F]KF was added a solution of the stannane precursor (5.0 mg, ˜0.009 mmol) in DMA (847 μL) followed by addition of a solution of [Cu(OTf)₂(py)₄] (0.2 M stock solution in DMA, 90 μL, 0.018 mmol) and pyridine (1 M stock solution in DMA, 63 μL, 0.063 mmol). The reaction mixture was then heated to 100° C. and allowed to stir for 15 mins. Upon cooling to 50° C., 2 mL of buffer (55% acetonitrile, 10 mM NH₄HCO₃, pH 10) was added and after stirring for an additional 1 min was transferred to an HPLC loop for injection and purification by semi-preparative chromatography (Gemini 5 μm NXC18 110 Å, 250×10, 4 mL/min). The product peak (retention time ˜18 min) was collected and diluted into 50 mL of MQ H₂O followed by trapping on a C18 extraction disk. The trapped product was washed with 10 ml of sterile water, eluted with 500 μL of EtOH and then rinsed with 4.0 mL of saline into the collection vial containing 5.5 mL of saline. The resulting 10 mL solution was then passed through a sterile filter into a sterile 10 mL dose vial. The identity and purity of [18F]3FN 1 was then confirmed using HPLC (Luna 5 μ C18 (2), 150×4.6, Buffer: 30% acetonitrile, 10 mM NH₄OAc, pH 5.0, 2 mL/min at 40° C.). [18F]3FN 1 was synthesized in >99% radiochemical purity with 9.4±2.2% activity yield (163.8±39.5 mCi in 10 mL, pH=5.0, MA=752.7±545.5 mCi/μmol, colorless solution, n=3; ˜72 min from EOB). Additionally, it was found that the formulated dose of [18F]3FN 1 was stable out to 4 h (>96% radiochemical purity).

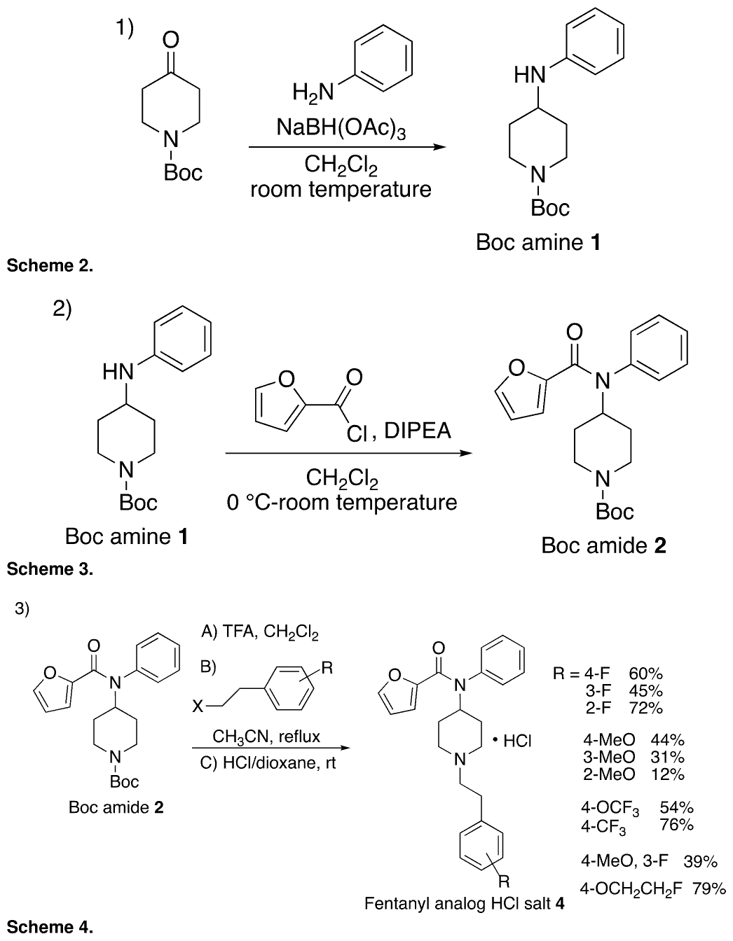

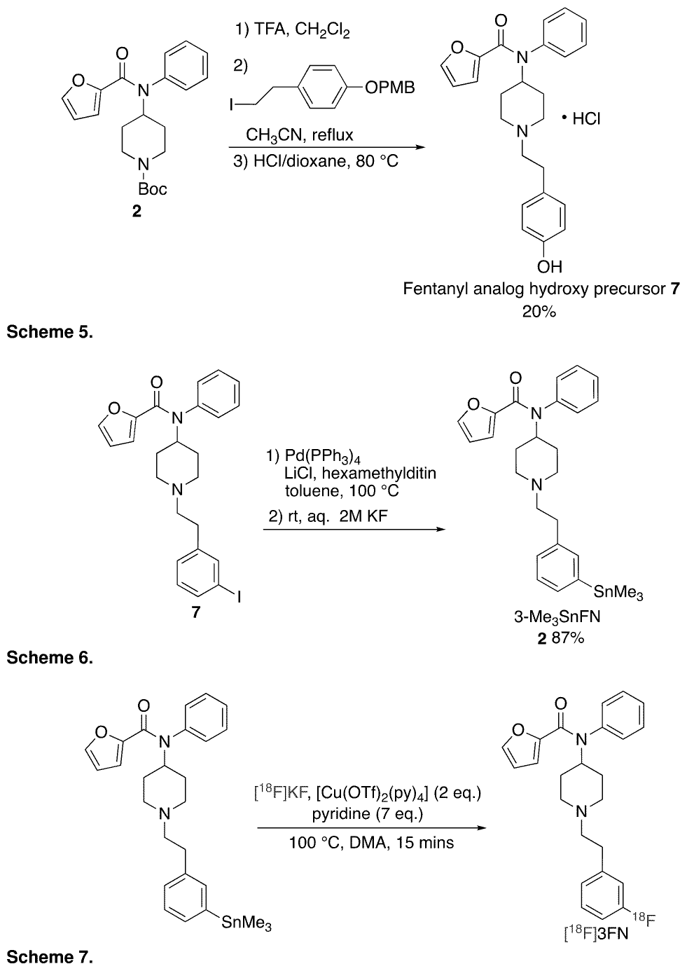

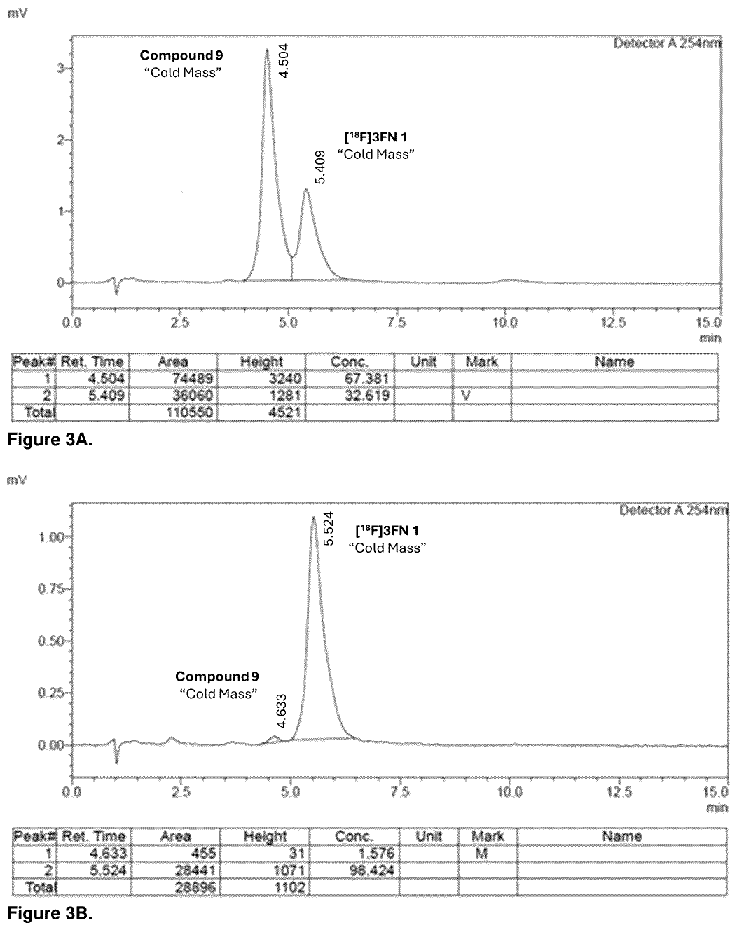

It should be noted that the main UV impurity in the chemical reaction is the presumed protodestannylated compound 9 (FIG. 3A). UV impurity 9 and [18F]3FN 1 have very similar retention times, but can be separated by semi-preparative HPLC when the column is allowed to equilibrate for an extended period (˜100 mins, 2 mL/min) hooked up to the synthesis module. This UV impurity has been found to account for ≥50% of the cold mass in the formulated dose when it is not fully separated from [18F]3FN 1 (FIG. 3B).

### Example 4—Evaluation of Compounds of the Disclosure

The series of fentanyl analogs 3FN 1 and 6a-f had their binding and efficacy profiles at the mu opioid receptor (MOR), delta opioid receptor (DOR) and kappa opioid receptor (KOR) (Tables 1, 2). Competitive displacement of [³H] diprenorphine at each receptor was carried out to evaluate binding at each of the receptors. A [³⁵S] GTPγS binding assay was used to determine the efficacy and potency of each compound. Percent stimulation values (Effect**) are derived from a comparison to a standard agonist for each receptor acting as a surrogate for efficacy with 0% stimulation indicating antagonist properties.

The fluoro series (3FN (1), 6a-b) was first evaluated and it was found that both 6a and 6b are partial agonists towards MOR (Table 1). Both exhibited some selectivity for MOR vs. DOR or KOR. Excitingly, 3FN 1 showed 0% stimulation in the [³⁵S] GTPγS binding assay indicating that it is an antagonist towards MOR. Additionally, it demonstrated MOR selectivity (Ki=2.4 vs. DOR Ki=50 and KOR Ki=52).

TABLE 1

| | MOR | | DOR | | KOR | |
|---|---|---|---|---|---|---|
| Compound | Kᵢ, nM* | Effect** | Kᵢ, nM* | Effect** | Kᵢ, nM* | Effect** |
| 6c | 1.4 ± 0.4 | partial agonist | 40 ± 7 | 0% | 18 ± 2 | 0% |
| | | (28%) | | | | |
| 3FN (1) | 2.4 ± 0.3 | antagonist | 50 ± 7 | 0% | 52 ± 2 | 0% |
| | | (0%) | | | | |
| 6e | 0.4 ± 0.1 | partial agonist | 11 ± 2 | 0% | 7 ± 2 | 0% |
| | | (34%) | | | | |
| fentanyl | 1.6 ± 0.4 | agonist | 82 ± 6 | 0% | 460 ± 100 | 0% |
| | | (98%) | | | | |

*Kᵢ is a measure of affinity for the receptor.
**Percent stimulation in the G protein activation assay compared to standard agonist (DAMGO at MOR, SNC80 at DOR, and U69593 at KOR). 0% is less than 10% observable stimulation up to a concentration of 10 μM.

Next, a methoxy series of fentanyl analogs (6c-e) was evaluated and two compounds were found to be partial agonists towards MOR (6d-e) (Table 2). Compound 6c exhibited antagonist characteristics. Unfortunately, it displayed similar binding affinities for MOR and KOR (Ki=7 nM vs. 8 nM) which was not desirable.

TABLE 2

| | MOR | | DOR | | KOR | |
|---|---|---|---|---|---|---|
| Compound | Kᵢ, nM* | Effect** | Kᵢ, nM* | Effect** | Kᵢ, nM* | Effect** |
| 6c | 7.0 ± 0.8 | antagonist | 100 ± 20 | 0% | 8 ± 1 | 0% |
| | | (0%) | | | | |
| 6d | 3.3 ± 0.9 | partial agonist | 22 ± 3 | 0% | 3.8 ± 0.2 | 0% |
| | | (16%) | | | | |
| 6e | 0.2 ± 0.1 | partial agonist | 1.5 ± 0.4 | 0% | 8 ± 2 | 0% |
| | | (54%) | | | | |
| fentanyl | 1.6 ± 0.4 | agonist | 82 ± 6 | 0% | 460 + 100 | 0% |
| | | (98%) | | | | |

*Ki is a measure of affinity for the receptor.
**Percent stimulation in the G protein activation assay compared to standard agonist (DAMGO at MOR, SNC80 at DOR, and U69593 at KOR). 0% is less than 10% observable stimulation up to a concentration of 10 μM.

### Example 5—In Vivo Uptake Studies of Compounds of the Disclosure

The uptake of compound [18F]F-3FN was evaluated in a monkey model across different administered doses (n=4; A: 5.27 mCi in 420 μL; B: 4.57 mCi in 440 μL; C: 4.04 mCi in 300 μL; D: 4.00 mCi in 280 μL). [18F]F-3FN was administered intravenously and PET imaging was performed at regular intervals, using an average administered dose of 4.47±0.59 mCi in 360±81.6 μL. The radioactivity was measured to determine the distribution of [18F]F-3FN relative to a control. The averaged radioactivity across the different doses administered and control data are presented in Tables 3 and 4, respectively.

TABLE 3

| Time [seconds] | | Time | Caudate-Putamen | | | | | Pituitary |
|---|---|---|---|---|---|---|---|---|
| Start | End | [min] | Left | Right | Thalamus | Cortex | Cerebellum | gland |
| | 0 | (min) | (Bq/cc) | (Bq/cc) | (Bq/cc) | (Bq/cc) | (Bq/cc) | (Bq/cc) |
| 0 | 60.00 | 0.50 | 12.53 | 11.76 | 9.32 | 9.88 | 9.56 | 14.07 |
| 60.00 | 120.00 | 1.50 | 83.33 | 72.61 | 69.30 | 54.19 | 83.96 | 133.10 |
| 120.00 | 180.00 | 2.50 | 121.51 | 120.00 | 115.85 | 91.33 | 126.76 | 172.49 |
| 180.00 | 240.00 | 3.50 | 122.70 | 139.63 | 121.50 | 97.09 | 129.53 | 184.78 |
| 240.00 | 300.00 | 4.50 | 153.79 | 150.13 | 146.76 | 107.34 | 134.26 | 206.39 |
| 300.00 | 450.00 | 6.25 | 141.37 | 143.47 | 127.66 | 102.70 | 124.80 | 179.82 |
| 450.00 | 600.00 | 8.75 | 133.03 | 129.88 | 117.95 | 95.22 | 118.42 | 164.59 |
| 600.00 | 900.00 | 12.50 | 155.92 | 154.02 | 118.41 | 96.83 | 100.05 | 156.89 |
| 900.00 | 1200.00 | 17.50 | 127.59 | 128.36 | 103.14 | 89.69 | 89.01 | 125.54 |
| 1200.00 | 1800.00 | 25.00 | 113.39 | 119.35 | 93.87 | 80.95 | 79.37 | 111.17 |
| 1800.00 | 2400.00 | 35.00 | 97.18 | 101.98 | 75.89 | 67.21 | 62.59 | 97.83 |
| 2400.00 | 3000.00 | 45.00 | 78.81 | 81.08 | 58.79 | 54.23 | 50.17 | 68.64 |
| 3000.00 | 3600.00 | 55.00 | 63.38 | 64.84 | 47.39 | 44.48 | 39.30 | 60.84 |
| 3600.00 | 4500.00 | 67.50 | 51.77 | 53.21 | 40.31 | 38.23 | 32.37 | 42.07 |
| 4500.00 | 5400.00 | 82.50 | 40.96 | 41.89 | 31.86 | 31.41 | 26.09 | 38.05 |

TABLE 4

| Time [seconds] | | Time | Caudate-Putamen | | | | | Pituitary |
|---|---|---|---|---|---|---|---|---|
| Start | End | [min] | Left | Right | Thalamus | Cortex | Cerebellum | gland |
| | 0 | (min) | (Bq/cc) | (Bq/cc) | (Bq/cc) | (Bq/cc) | (Bq/cc) | (Bq/cc) |
| 0 | 60.00 | 0.50 | 15.10 | 16.50 | 15.99 | 16.38 | 13.73 | 15.04 |
| 60.00 | 120.00 | 1.50 | 80.40 | 79.21 | 77.60 | 78.46 | 78.31 | 80.22 |
| 120.00 | 180.00 | 2.50 | 110.54 | 114.54 | 104.77 | 105.35 | 111.09 | 115.41 |
| 180.00 | 240.00 | 3.50 | 103.84 | 112.14 | 105.04 | 98.19 | 102.14 | 132.98 |
| 240.00 | 300.00 | 4.50 | 129.48 | 133.35 | 116.98 | 116.01 | 116.16 | 122.01 |
| 300.00 | 450.00 | 6.25 | 128.09 | 126.88 | 120.48 | 109.58 | 110.07 | 141.35 |
| 450.00 | 600.00 | 8.75 | 126.29 | 122.94 | 115.96 | 101.49 | 103.87 | 149.43 |
| 600.00 | 900.00 | 12.50 | 106.19 | 108.48 | 100.96 | 91.32 | 82.64 | 141.23 |
| 900.00 | 1200.00 | 17.50 | 85.29 | 86.44 | 81.18 | 72.48 | 63.95 | 104.32 |
| 1200.00 | 1800.00 | 25.00 | 62.37 | 62.77 | 59.89 | 53.23 | 45.50 | 67.24 |
| 1800.00 | 2400.00 | 35.00 | 44.28 | 44.61 | 41.47 | 37.13 | 31.46 | 45.20 |
| 2400.00 | 3000.00 | 45.00 | 33.83 | 34.59 | 33.19 | 28.58 | 25.28 | 37.21 |
| 3000.00 | 3600.00 | 55.00 | 28.55 | 27.72 | 27.00 | 24.56 | 20.93 | 29.25 |
| 3600.00 | 4500.00 | 67.50 | 23.89 | 24.13 | 22.12 | 21.83 | 17.95 | 25.43 |
| 4500.00 | 5400.00 | 82.50 | 20.92 | 19.90 | 18.87 | 18.55 | 15.97 | 26.54 |

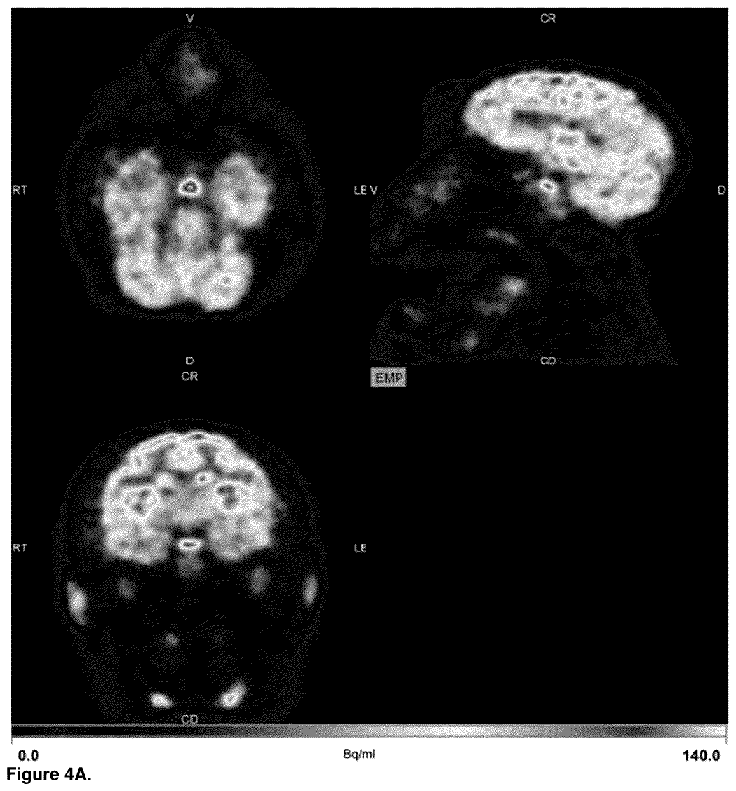

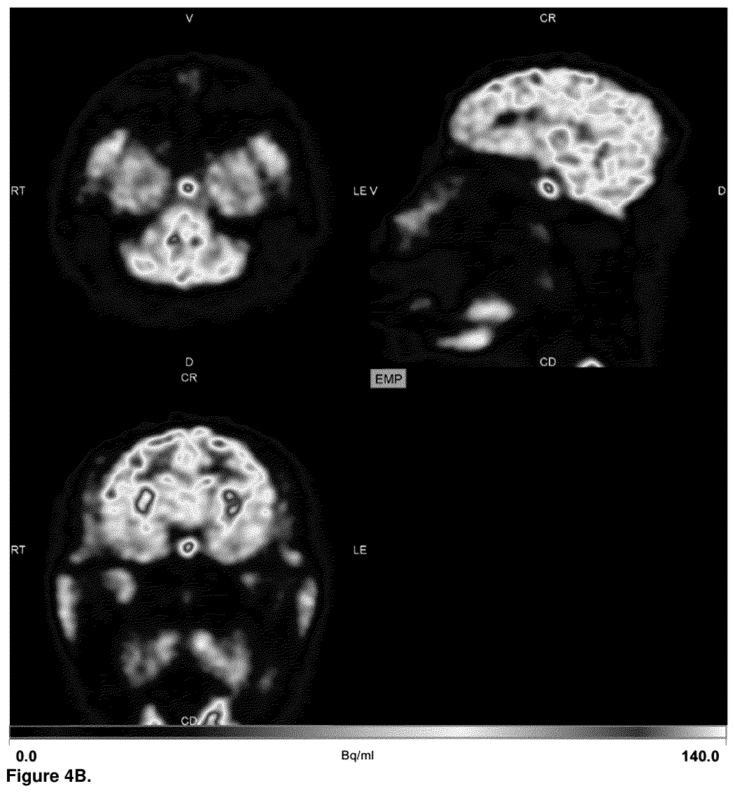

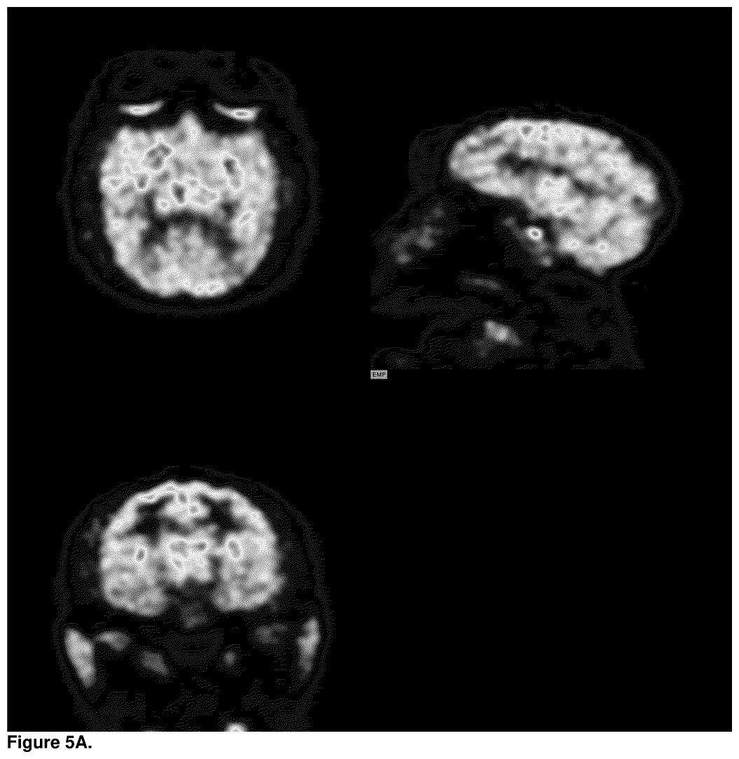

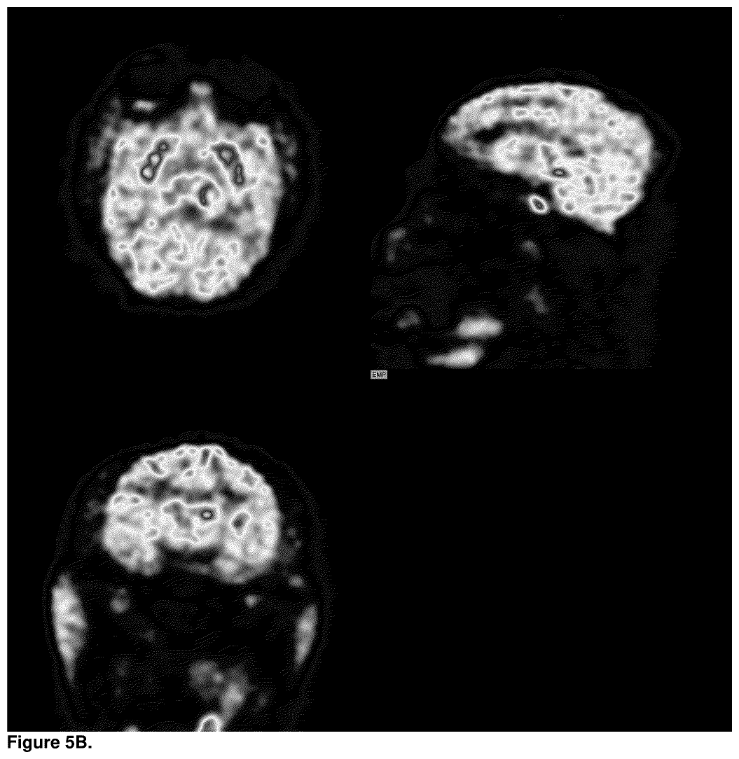

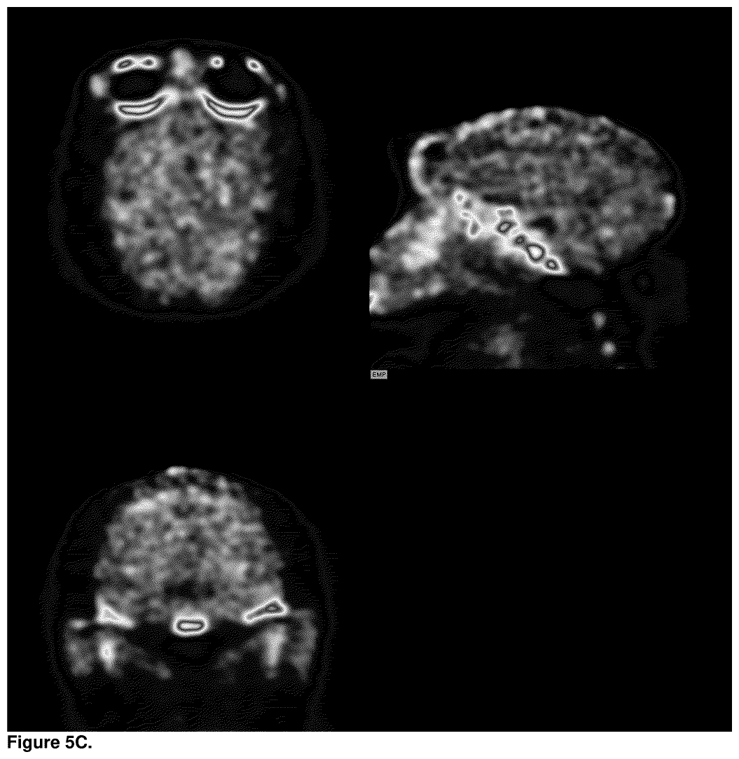

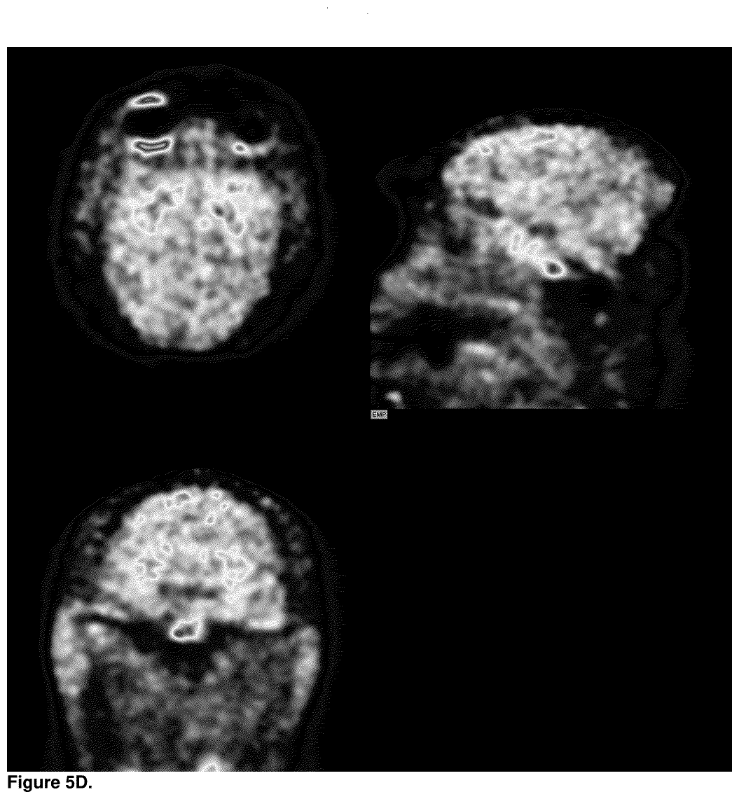

Imaging of the monkey model was performed at predetermined times to evaluate the uptake of [18F]3FN in the monkey brain. As shown in FIG. 4A and 4B, imaging indicated that [18F]3FN was taken up into the pituitary gland after 15 min., as compared to the control image. The distribution of [18F]3FN was also investigated across the caudate-putamen (left and right), thalamus, cortex, cerebellum, in addition to the pituitary gland and is shown in FIGS. 5A-5D. The [18F]3FN was also seen to be taken up in a range of brain tissues, as compared to the control.

### Aspects of the Disclosure

Aspect 1. A compound, or pharmaceutically acceptable salt thereof, having a structure of Formula (I):

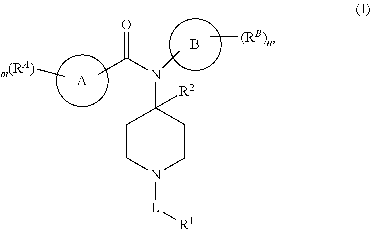

wherein:

each m and n are independently 0, 1, 2, or 3;

ring A is a 4-8 membered heterocycle, or 5-10 membered heteroaryl, wherein the heterocycle and heteroaryl each comprises 1, 2, or 3 ring heteroatoms selected from N, O, and S;

each R^{A}, when present, is C₁₋₃alkyl or C₁haloalkyl;

ring B is a C₆₋₁₀aryl, or 5-10 membered heteroaryl, wherein the heteroaryl comprises 1, 2, or 3 ring heteroatoms selected from N, O, and S;

each R^{B}, when present, is C₁₋₃alkyl or C₁haloalkyl;

L is C₂₋₆alkylene;

R¹ is halo, C₁₋₆alkoxyl, C₁₋₆haloalkyl, C₁₋₆haloalkoxyl, C₆₋₁₀aryl, 4-8 membered heterocycle, or 5-10 membered heteroaryl, wherein the heterocycle and heteroaryl each comprises 1, 2, or 3 ring heteroatoms selected from N, O, and S, and when R¹ is C₆₋₁₀aryl, 4-8 membered heterocycle or 5-10 membered heteroaryl, R¹ is substituted with 1 to 3 R^{1A} groups;

each R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X;

R² is H, —C(O)O—C₁₋₃alkyl, —C(O)O—C₁₋₃alkylene—X, or —C(O)O—C₁₋₃alkylene—LG;

X is a radioisotope; and

LG is a leaving group;

with the proviso that: only one X is present or X is absent.

Aspect 2. The compound or salt of any one of the proceeding aspects, wherein ring A is a 5-10 membered heteroaryl, and comprises 1, 2, or 3 ring heteroatoms selected from N, O, and S.

Aspect 3. The compound or salt of any one of the proceeding aspects, wherein ring A is pyrrolyl, pyrazolidinyl, imidazolyl, furanyl, thiophenyl, oxazolyl, isoxazolyl, thiazolyl, and isothiazolyl.

Aspect 4. The compound or salt of any one of the proceeding aspects, having a structure of Formula (Ia):

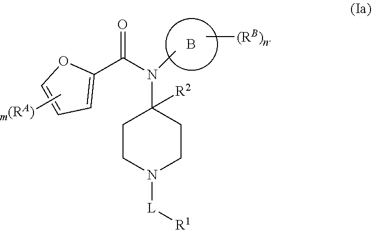

Aspect 5. The compound or salt of any one of the proceeding aspects, wherein m is 0 or 1.

Aspect 6. The compound or salt of any one of the proceeding aspects, wherein R^{A}, when present, is C₁alkyl or C₁haloalkyl.

Aspect 7. The compound or salt of any one of the proceeding aspects, wherein R^{A}, when present, is C₁alkyl.

Aspect 8. The compound or salt of any one of the proceeding aspects, wherein R¹ is halo, C₁₋₆alkoxyl, C₁₋₆haloalkyl, or C₁₋₆haloalkoxyl.

Aspect 9. The compound or salt of any one of the proceeding aspects, wherein R¹ is C₆₋₁₀aryl, 4-8 membered heterocycle or 5-10 membered heteroaryl and R¹ is substituted with 0 to 3 R^{1A} groups.

Aspect 10. The compound or salt of any one of the proceeding aspects, wherein R¹ is C₆₋₁₀aryl.

Aspect 11. The compound or salt of any one of the proceeding aspects, having a structure of Formula (Ib):

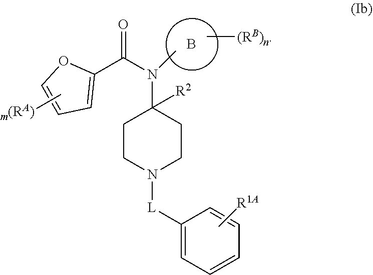

Aspect 12. The compound or salt of any one of the proceeding aspects, wherein L is C₂₋₄alkylene.

Aspect 13. The compound or salt of any one of the proceeding aspects, wherein L is C₂alkylene.

Aspect 14. The compound or salt of any one of the proceeding aspects, having a structure of Formula (Ic):

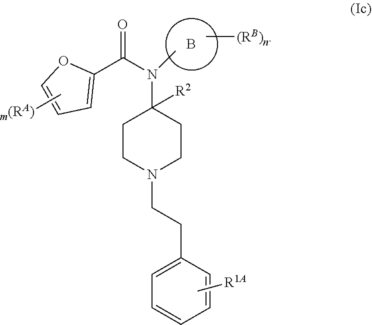

Aspect 15. The compound or salt of any one of the proceeding aspects, wherein R^{1A} is halo, C₁₋₃alkoxyl, C₁₋₃haloalkoxyl, X, or LG.

Aspect 16. The compound or salt of any one of the proceeding aspects, wherein R^{1A} is halo, X, or LG.

Aspect 17. The compound or salt of any one of the proceeding aspects, wherein R^{1A} is C₁₋₃alkoxyl or C₁₋₃haloalkoxyl, and is substituted with X.

Aspect 18. The compound or salt of any one of the proceeding aspects, having a structure of Formula (Id):

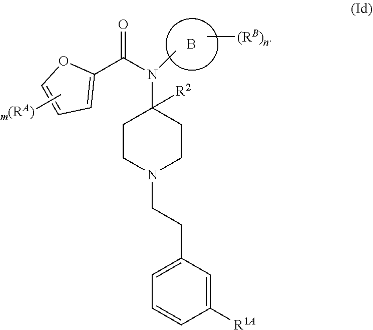

Aspect 19. The compound or salt of any one of the proceeding aspects, wherein R² is —C(O)O—C₁₋₃alkyl, —C(O)O—C₁₋₃alkylene—X, or —C(O)O—C₁₋₃alkylene—LG.

Aspect 20. The compound or salt of any one of the proceeding aspects, wherein R² is —C(O)O—C₁alkylene—X or —C(O)O—C₁₋₃alkylene—LG.

Aspect 21. The compound or salt of any one of the proceeding aspects, wherein ring B is C₆₋₁₀aryl.

Aspect 22. The compound or salt of any one of the proceeding aspects, having a structure of Formula (Ie):

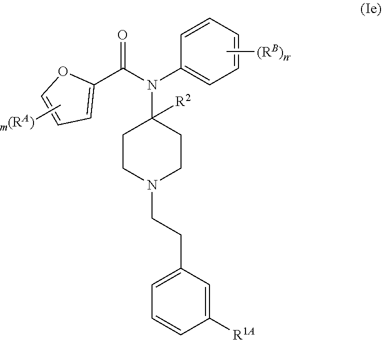

Aspect 23. The compound or salt of any one of the proceeding aspects, wherein n is 0.

Aspect 24. The compound or salt of any one of the proceeding aspects, wherein ring B is pyridyl.

Aspect 25. The compound or salt of any one of the proceeding aspects, having a structure of Formula (If):

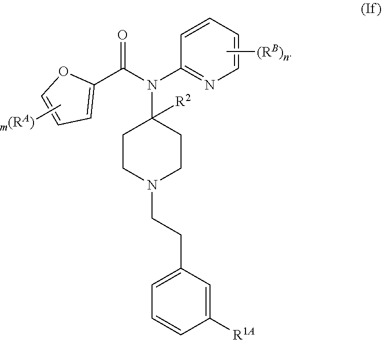

Aspect 26. The compound or salt of any one of the proceeding aspects, wherein n is 1.

Aspect 27. The compound or salt of any one of the proceeding aspects, wherein R^{B}, when present, is C₁alkyl or C₁haloalkyl.

Aspect 28. The compound or salt of any one of the proceeding aspects, wherein R^{B}, when present, is C₁alkyl.

Aspect 29. The compound or salt of any one of the proceeding aspects, wherein R² is —C(O)O—C₁alkylene—X and the radioisotope is ¹³C.

Aspect 30. The compound or salt of any one of the proceeding aspects, wherein R^{1A} is X and the radioisotope is ¹⁸F.

Aspect 31. The compound or salt of any one of the proceeding aspects, wherein R^{1A} is LG.

Aspect 32. The compound or salt of any one of the proceeding aspects, wherein R² is —C(O)O—C₁₋₃alkylene—LG.

Aspect 33. The compound or salt of aspect 30 or 31, wherein LG is OH, SnMe₃, SnBu₃, or

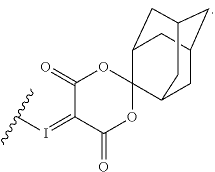 [SMILES: *I=C1C(=O)OC2(OC1=O)C1CC3CC(C1)CC2C3]

Aspect 34. A compound, or pharmaceutically acceptable salt thereof, having a structure of Formula (II):

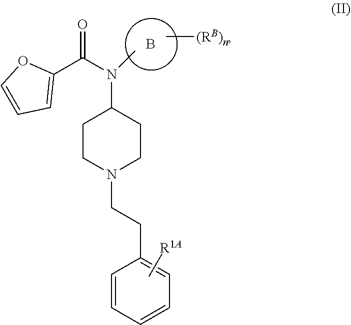

wherein:

n is 0 or 1;

ring B is phenyl or pyridyl;

each R^{B}, when present, is C₁alkyl or C₁haloalkyl;

R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X,

X is a radioisotope; and

LG is a leaving group

with the proviso that exclusively one X is present.

Aspect 35. The compound or salt of any one of the proceeding aspects, having a structure of Formula (IIa):

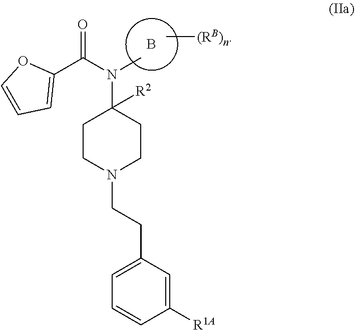

Aspect 36. The compound or salt of any one of the proceeding aspects, wherein R^{1A} is halo, C₁₋₃alkoxyl, C₁₋₃haloalkoxyl, X, or LG.

Aspect 37. The compound or salt of any one of the proceeding aspects, wherein R^{1A} is halo, X, or LG.

Aspect 38. The compound or salt of any one of the proceeding aspects, wherein ring B is phenyl.

Aspect 39. The compound or salt of any one of the proceeding aspects, wherein n is 0.

Aspect 40. The compound or salt of any one of the proceeding aspects, having a structure of Formula (IIb):

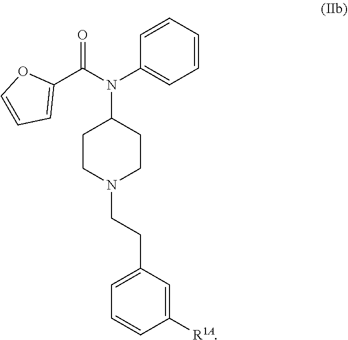

Aspect 41. The compound or salt of any one of the proceeding aspects, wherein ring B is pyridyl.

Aspect 42. The compound or salt of any one of the proceeding aspects, wherein n is 1.

Aspect 43. The compound or salt of any one of the proceeding aspects, having a structure of Formula (IIc):

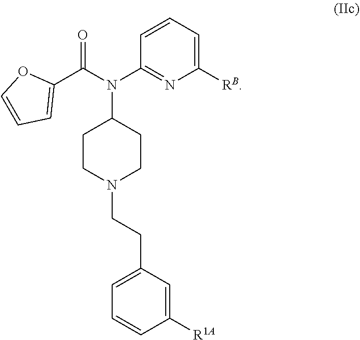

Aspect 44. The compound or salt of any one of the proceeding aspects, wherein R^{B}, when present, is C₁alkyl.

Aspect 45. The compound or salt of any one of the proceeding aspects, wherein R^{1A} is C₁₋₃alkyl or C₁₋₃alkoxyl, R^{1A} is substituted with X, and the radioisotope is ¹¹C.

Aspect 46. The compound or salt of any one of the proceeding aspects, wherein R^{1A} is C₁₋₃alkoxyl substituted with X, and the radioisotope is ¹⁸F.

Aspect 47. The compound or salt of any one of the proceeding aspects, wherein R^{1A} is X and the radioisotope is ¹⁸F.

Aspect 48. The compound or salt of any one of the proceeding aspects, wherein LG is OH, SnMe₃, SnBu₃, or

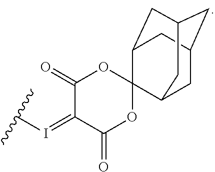 [SMILES: *I=C1C(=O)OC2(OC1=O)C1CC3CC(C1)CC2C3]

Aspect 49. A compound, or pharmaceutically acceptable salt thereof, having a structure selected from

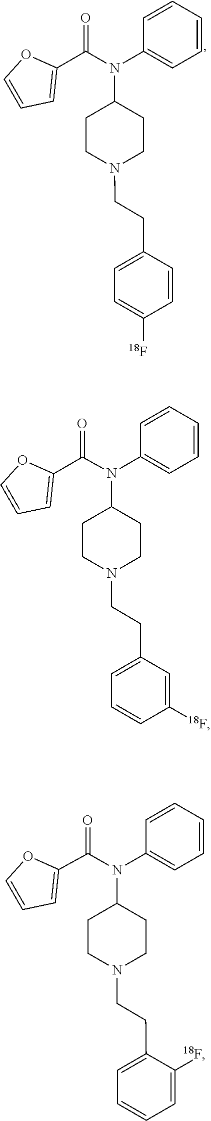

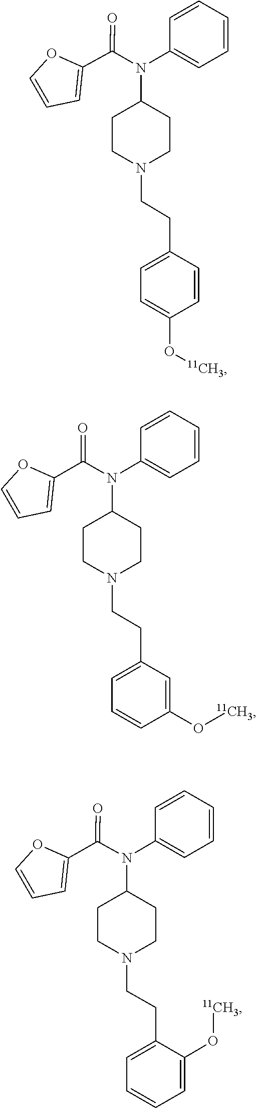

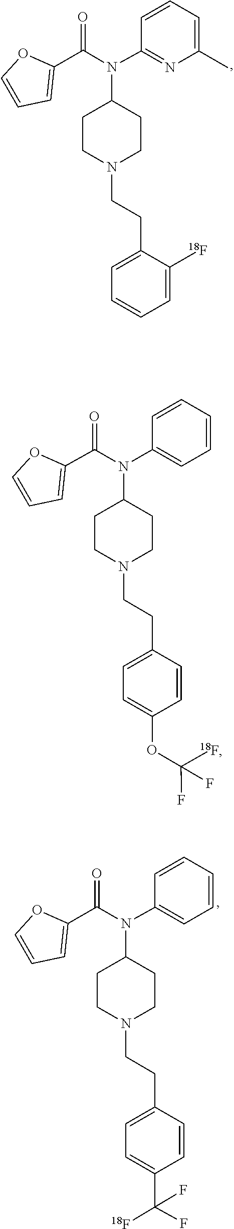

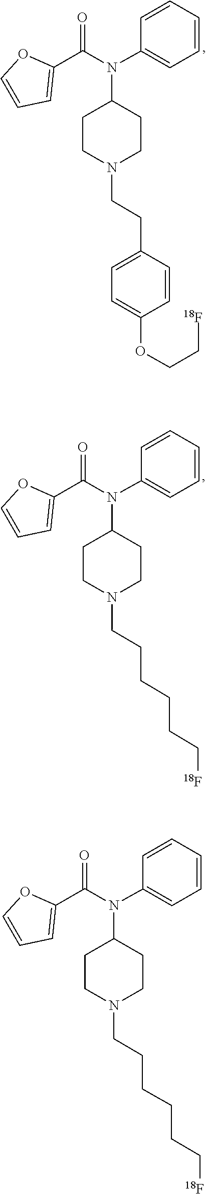 [SMILES: O=C(c1ccco1)N(c1ccccc1)C1CCN(CCc2ccc(OCC[18F])cc2)CC1 ; O=C(c1ccco1)N(c1ccccc1)C1CCN(CCCCCC[18F])CC1 ; O=C(c1ccco1)N(c1ccccc1)C1CCN(CCCCCC[18F])CC1]

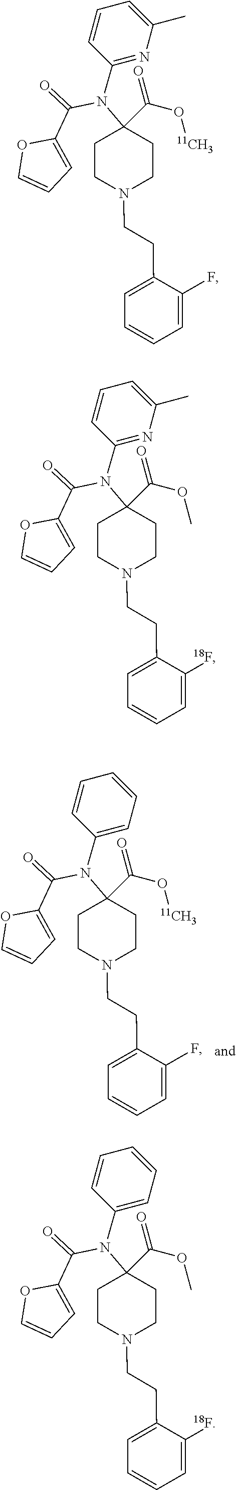

Aspect 50. The compound or salt of aspect 48, having a structure selected from

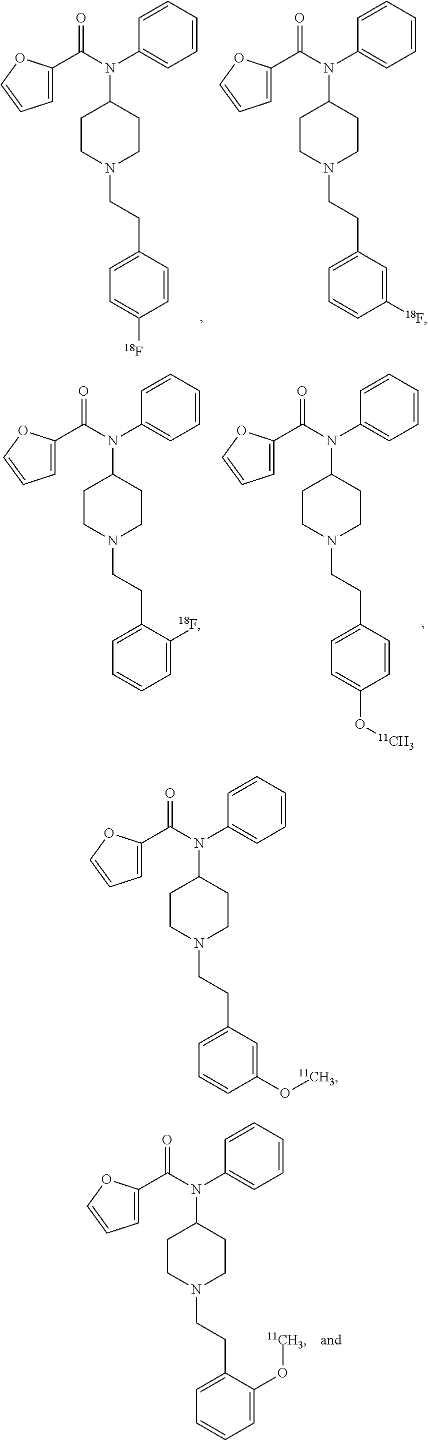

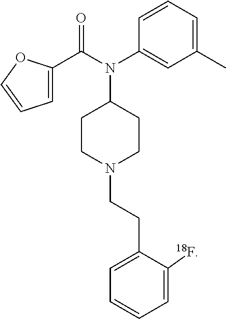

Aspect 51. The compound or salt of aspect 48, having the following structure

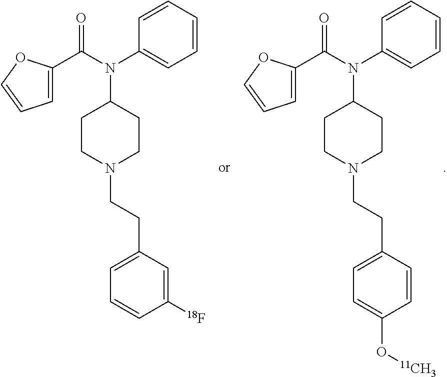 [SMILES: O=C(c1ccco1)N(c1ccccc1)C1CCN(CCc2cccc([18F])c2)CC1 ; [11CH3]Oc1ccc(CCN2CCC(N(C(=O)c3ccco3)c3ccccc3)CC2)cc1]

Aspect 52. A compound, or pharmaceutically acceptable salt thereof, having a structure selected from

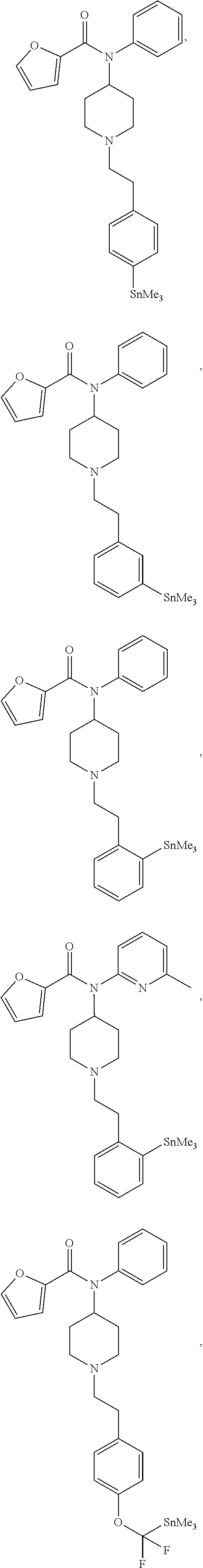

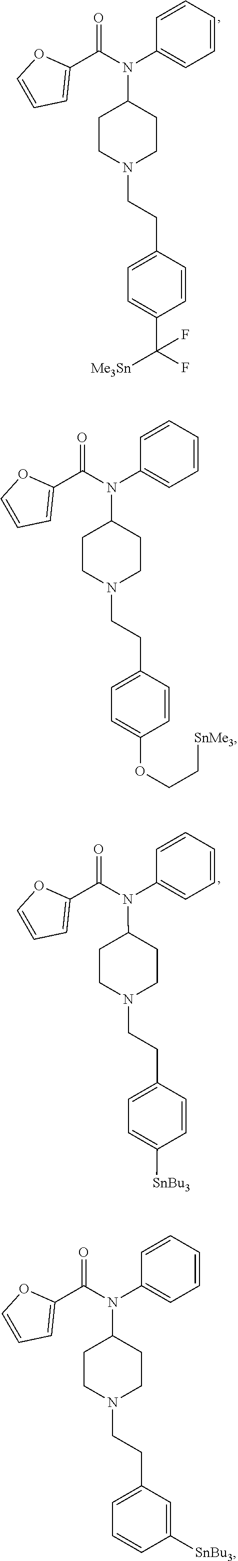

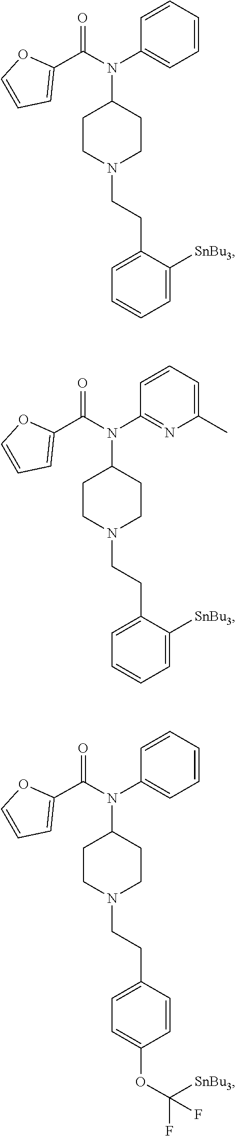

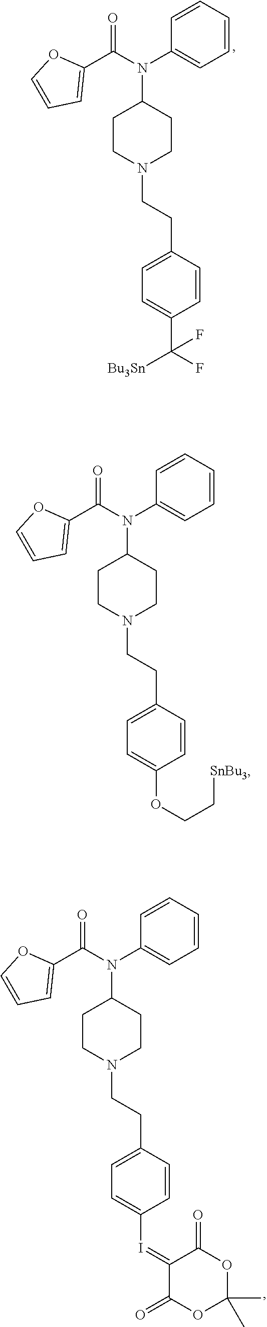

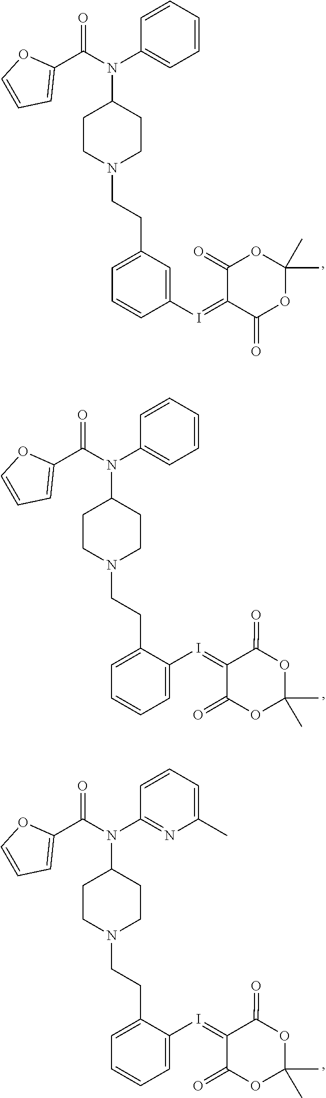 [SMILES: CC1(C)OC(=O)C(=Ic2cccc(CCN3CCC(N(C(=O)c4ccco4)c4ccccc4)CC3)c2)C(=O)O1 ; CC1(C)OC(=O)C(=Ic2ccccc2CCN2CCC(N(C(=O)c3ccco3)c3ccccc3)CC2)C(=O)O1 ; Cc1cccc(N(C(=O)c2ccco2)C2CCN(CCc3ccccc3I=C3C(=O)OC(C)(C)OC3=O)CC2)n1]

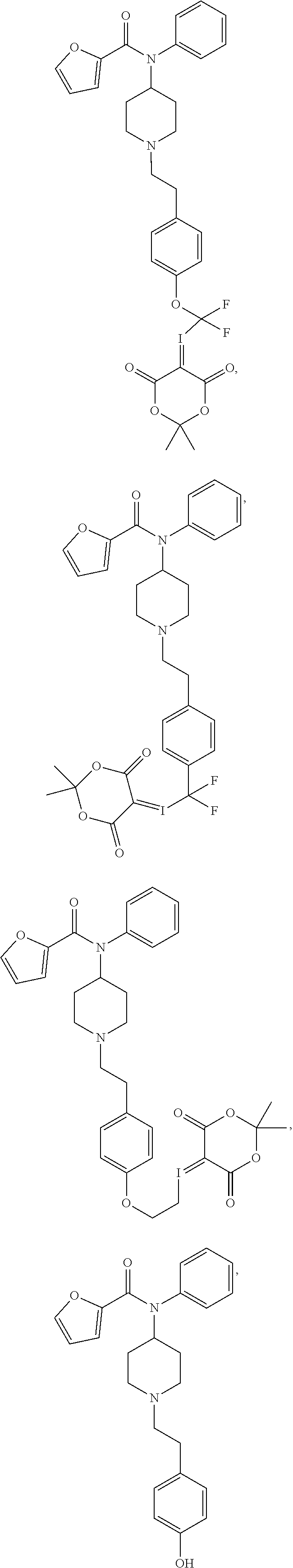

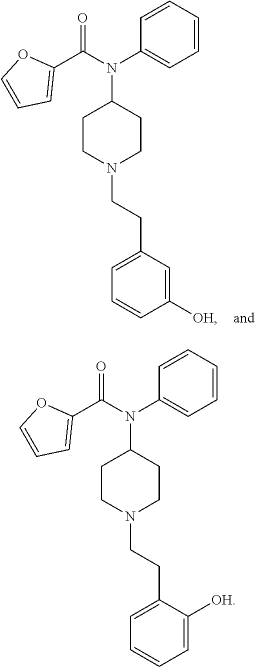

Aspect 53. The compound or salt of aspect 50, having a structure selected from

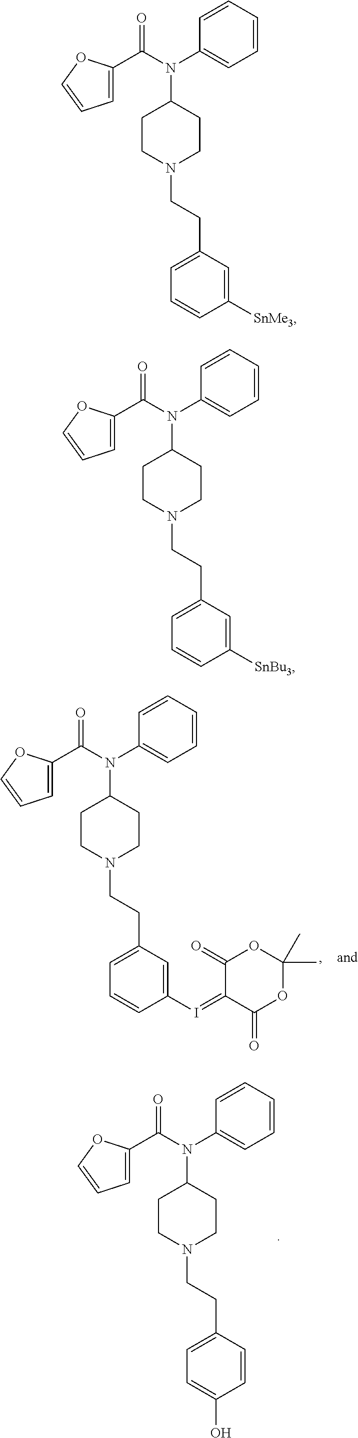

Aspect 54. A method comprising

administering to a subject the compound of aspects 1, 33, or 49; and

subjecting the subject to an imaging modality.

Aspect 55. The method of aspect 53, wherein the imaging modality is selected from the group consisting of positron emission tomography (PET), positron emission tomography/computed tomography (PET/CT), positron emission tomography/magnetic resonance imaging (PET/MRI), single-photon emission computerized tomography (SPECT), and single-photon emission computerized tomography/computed tomography (SPECT/CT).

Aspect 56. The method of aspect 53 or 54, wherein the imaging modality is positron emission tomography (PET).

What is claimed is:

1. A compound, or pharmaceutically acceptable salt thereof, having a structure of Formula (I): 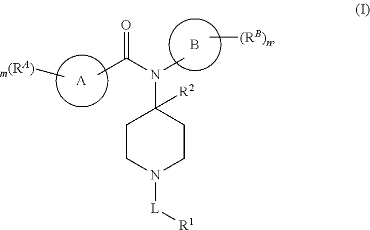
wherein:
each m and n are independently 0, 1, 2, or 3;
ring A is a 4-8 membered heterocycle, or 5-10 membered heteroaryl, wherein the heterocycle and heteroaryl each comprises 1, 2, or 3 ring heteroatoms selected from N, O, and S;
each R^{A}, when present, is C₁₋₃alkyl or C₁haloalkyl;
ring B is a C₆₋₁₀aryl, or 5-10 membered heteroaryl, wherein the heteroaryl comprises 1, 2, or 3 ring heteroatoms selected from N, O, and S;
each R^{B}, when present, is C₁₋₃alkyl or C₁haloalkyl;
L is C₂₋₆alkylene;
R¹ is halo, C₁₋₆alkoxyl, C₁₋₆haloalkyl, C₁₋₆haloalkoxyl, C₆₋₁₀aryl, 4-8 membered heterocycle, or 5-10 membered heteroaryl, wherein the heterocycle and heteroaryl each comprises 1, 2, or 3 ring heteroatoms selected from N, O, and S, and when R¹ is C₆₋₁₀aryl, 4-8 membered heterocycle or 5-10 membered heteroaryl, R¹ is substituted with 1 to 3 R^{1A} groups;
each R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X;
R² is H, —C(O)O—C₁₋₃alkyl, —C(O)O—C₁₋₃alkylene—X, or —C(O)O—C₁₋₃alkylene—LG;
X is a radioisotope; and
LG is a leaving group;
with the proviso that: only one X is present or X is absent.

2. The compound or salt of claim 1, wherein ring A is a 5-10 membered heteroaryl, and comprises 1, 2, or 3 ring heteroatoms selected from N, O, and S.

3. The compound or salt of claim 1, having a structure of Formula (Ia):

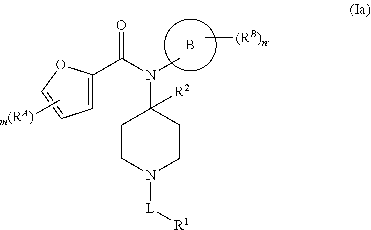

4. The compound or salt of claim 3, wherein R¹ is C₆₋₁₀aryl, 4-8 membered heterocycle or 5-10 membered heteroaryl and R¹ is substituted with 0 to 3 R^{1A} groups.

5. The compound or salt of claim 1, having a structure of Formula (Ib):

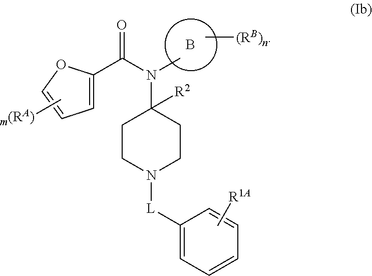

6. The compound or salt of claim 1, having a structure of Formula (Ic):

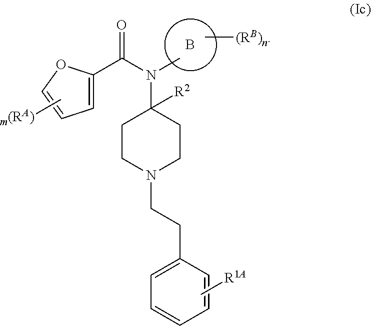

7. The compound or salt of claim 6, wherein R^{1A} is halo, C₁₃alkoxyl, C₁₋₃haloalkoxyl, X, or LG, and the C₁₋₃alkoxyl or C₁₋₃haloalkoxyl, and is substituted with 0 or 1 X.

8. The compound or salt of claim 1, having a structure of Formula (Id):

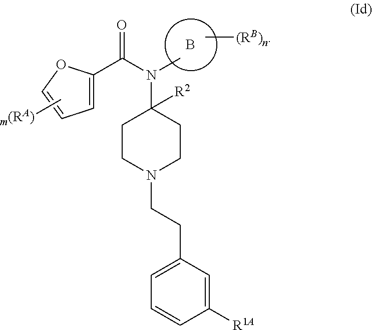

9. The compound or salt of claim 1, having a structure of Formula (Ie):

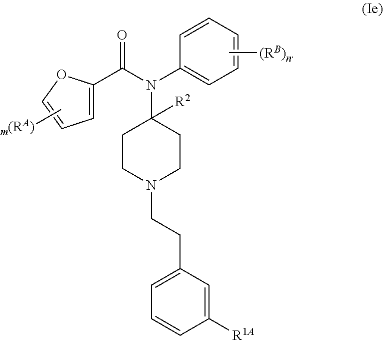

10. The compound or salt of claim 1, having a structure of Formula (If):

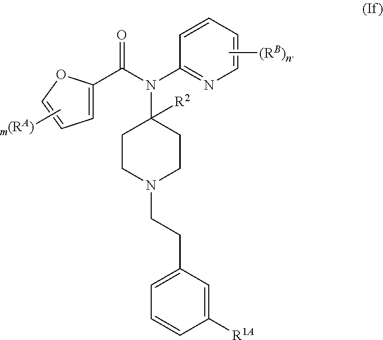

11. The compound or salt of claim 10, wherein (i) R² is —C(O)O-C₁alkylene-X and the radioisotope is ¹³C and/or (ii) R^{1A} is X and the radioisotope is ¹⁸F.

12. A compound, or pharmaceutically acceptable salt thereof, having a structure of Formula (II): 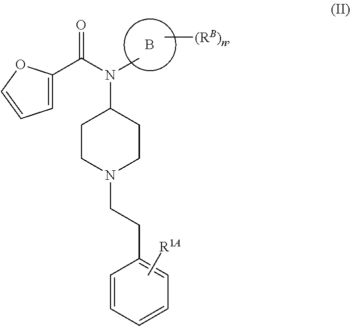
wherein:
n is 0 or 1;
ring B is phenyl or pyridyl;
each R^{B}, when present, is C₁alkyl or C₁haloalkyl;
R^{1A} is halo, C₁₋₃alkyl, C₁₋₃alkoxyl, C₁₋₃haloalkyl, C₁₋₃haloalkoxyl, X, or LG, and when C₁₋₃alkyl or C₁₋₃alkoxyl, R^{1A} can be substituted with X,
X is a radioisotope; and
LG is a leaving group
with the proviso that exclusively one X is present.

13. The compound or salt of claim 12, having a structure of Formula (IIa):

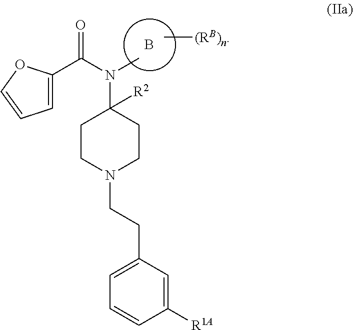

14. The compound or salt of claim 12, having a structure of Formula (IIb):

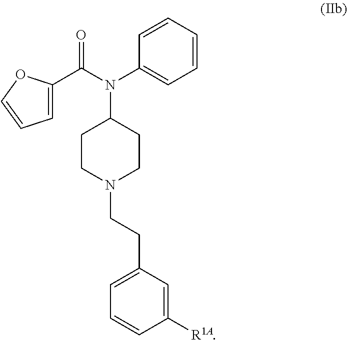

15. The compound or salt of claim 12, having a structure of Formula (IIc):

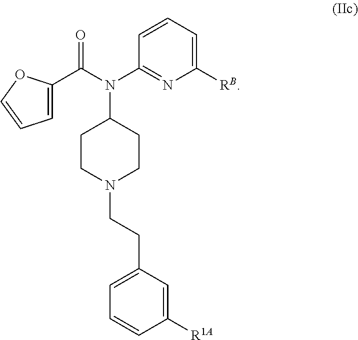

16. The compound or salt of claim 1, wherein R^{1A} is C₁₋₃alkyl or C₁₋₃alkoxyl, R^{1A} is substituted with X, and the radioisotope is ¹¹C.

17. The compound or salt of claim 1, wherein R¹⁴ is X or C₁₋₃alkoxyl substituted with X, and the radioisotope is ¹⁸F.

18. The compound or salt of claim 1, wherein LG is OH, SnMe₃, SnBu₃, or

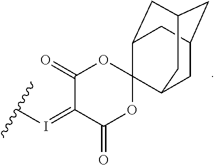 [SMILES: *I=C1C(=O)OC2(OC1=O)C1CC3CC(C1)CC2C3]

19. A compound, or pharmaceutically acceptable salt thereof, having a structure selected from

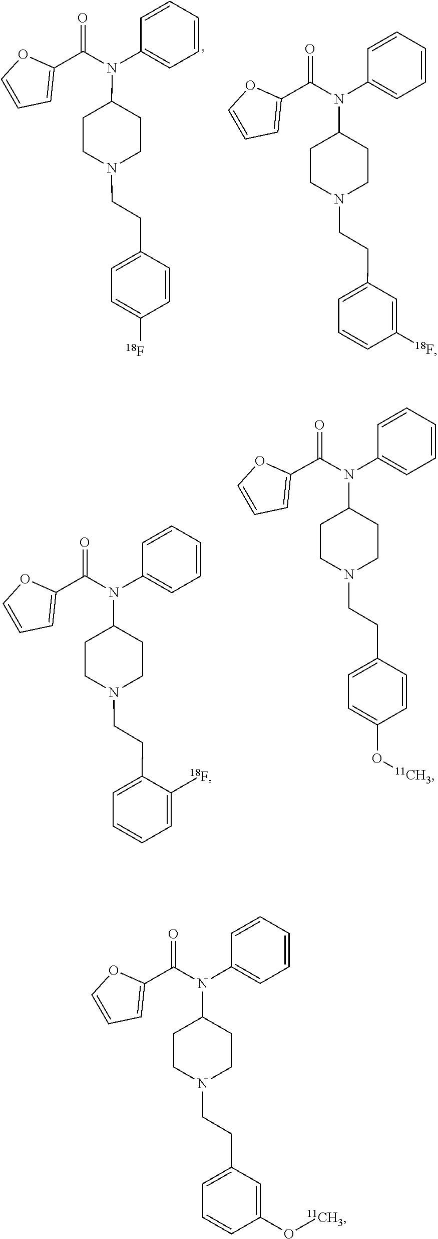

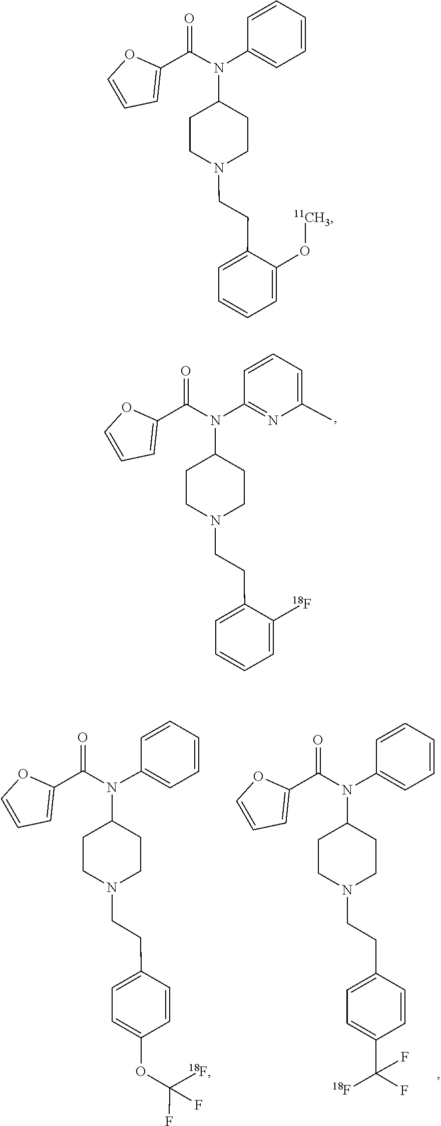

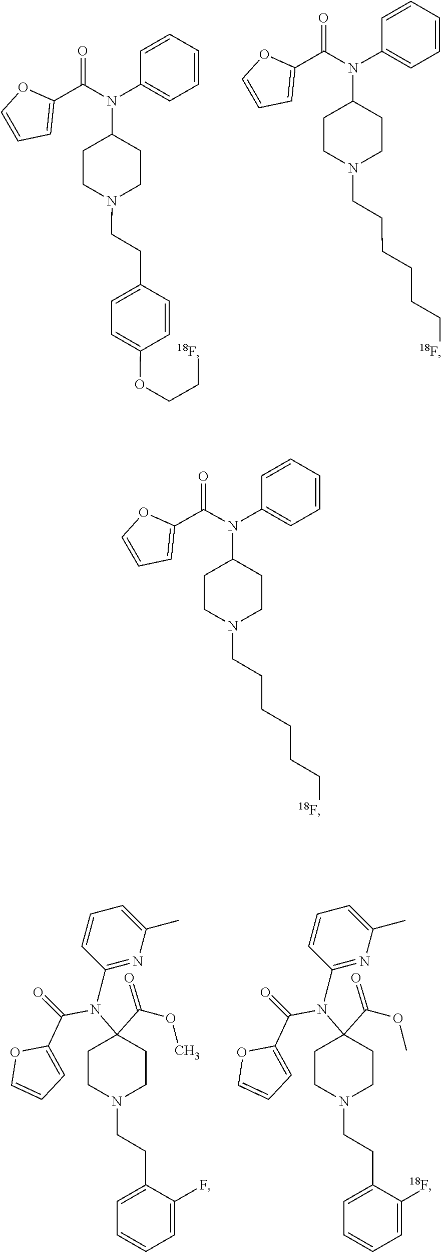

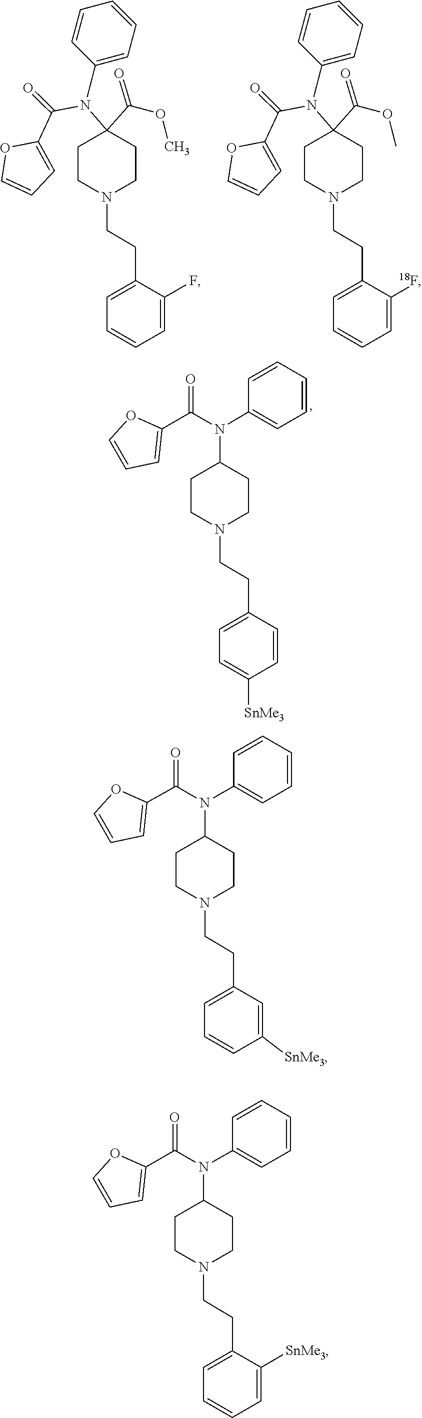

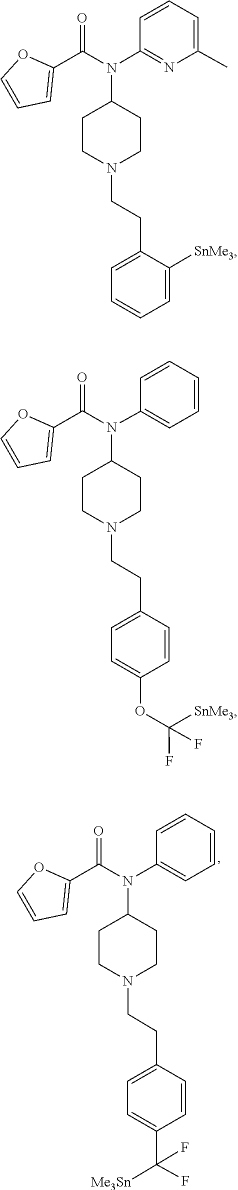

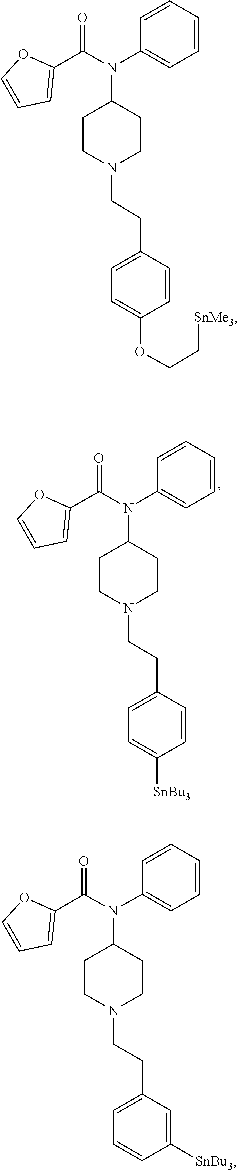

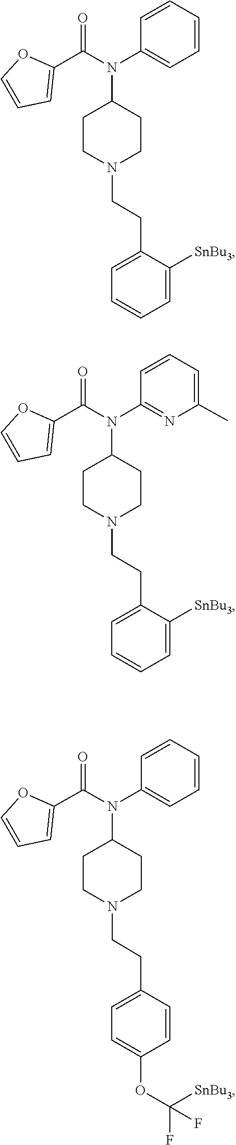

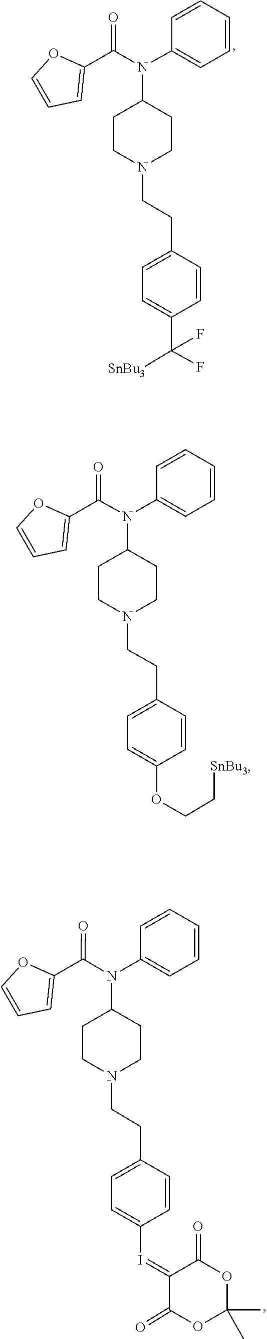

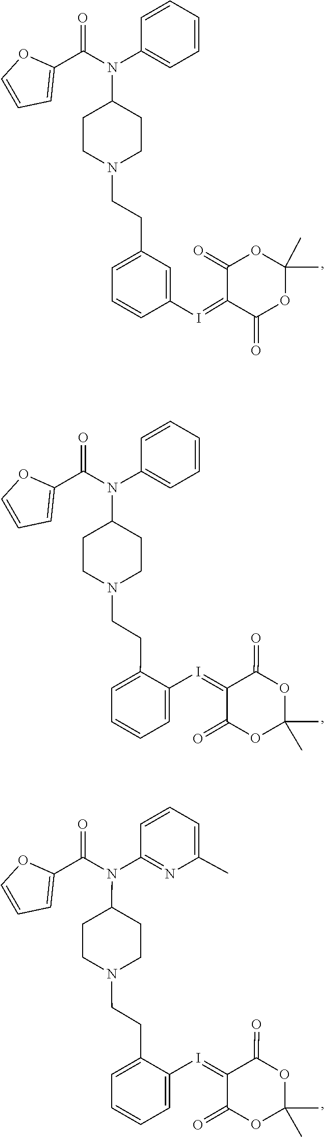 [SMILES: CC1(C)OC(=O)C(=Ic2cccc(CCN3CCC(N(C(=O)c4ccco4)c4ccccc4)CC3)c2)C(=O)O1 ; CC1(C)OC(=O)C(=Ic2ccccc2CCN2CCC(N(C(=O)c3ccco3)c3ccccc3)CC2)C(=O)O1 ; Cc1cccc(N(C(=O)c2ccco2)C2CCN(CCc3ccccc3I=C3C(=O)OC(C)(C)OC3=O)CC2)n1]

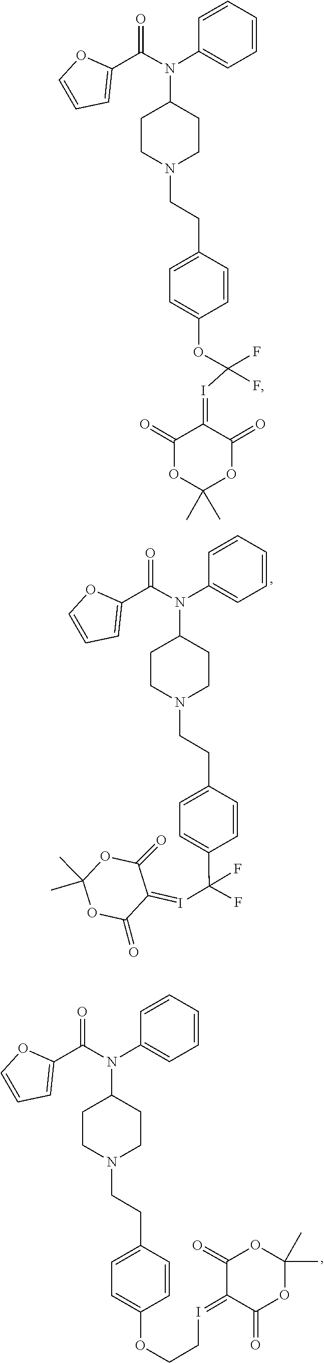

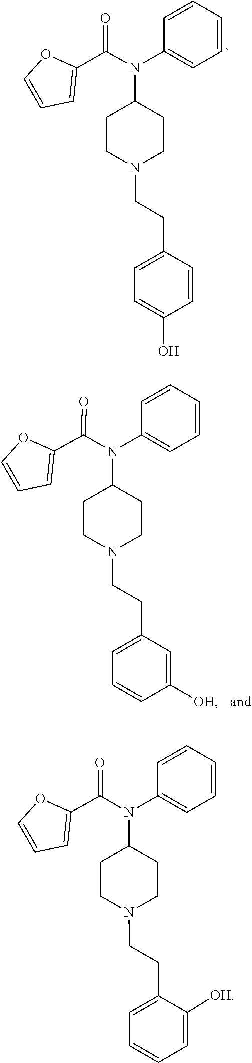

20. A method comprising
administering to a subject the compound of claims 1; and
subjecting the subject to an imaging modality selected from the group consisting of positron emission tomography (PET), positron emission tomography/computed tomography (PET/CT), positron emission tomography/magnetic resonance imaging (PET/MRI), single-photon emission computerized tomography (SPECT), and single-photon emission computerized tomography/computed tomography (SPECT/CT).
